(19) United States
(12) Patent Application Publication
(10) Pub. No.: US 2024/0358945 A1
(43) Pub. Date: Oct. 31, 2024

(54) METHODS AND APPARATUS FOR DETECTING SLEEP

(71) Applicant: Ectosense nv, Sluisstraat, Leuven (BE)

(72) Inventors: Frederik Roger Anne MASSIE, Wilsele (BE); Bart VAN PEE, Leuven (BE); Steven Elsa Louis VITS, St-Gillis (BE)

(73) Assignee: Ectosense nv, Sluisstraat, Leuven (BE)

(21) Appl. No.: 18/560,307

(22) PCT Filed: May 11, 2022
(86) PCT No.: PCT/EP2022/062807

§ 371 Date: Nov. 10, 2023

(30) Foreign Application Priority Data
May 12, 2021 (EP) 21173562.6

(51) Int. Cl. A61M 16/00; A61M 16/06; A61M 16/08; A61M 16/10; G16H 50/20; G16H 50/30

(52) U.S. Cl. CPC A61M 16/024; A61M 16/0003; A61M 16/0066; A61M 16/0683; A61M 16/0875; A61M 16/1075; G16H 50/20; G16H 50/30; A61M 2016/0027; A61M 2016/0033; A61M 2202/0007; A61M 2202/0208; A61M 2205/103; A61M 2205/3303; A61M 2205/3327; A61M 2205/3334; A61M 2205/3351; A61M 2205/3365; A61M 2205/3553; A61M 2205/3561; A61M 2205/42; A61M 2205/502; A61M 2205/581; A61M 2205/583; A61M 2205/75; A61M 2210/0606; A61M 2210/0618; A61M 2230/04; A61M 2230/20; A61M 2230/42; A61M 2230/63

(57) ABSTRACT

Apparatus and methods detect sleep disordering events. The apparatus may be configured to access one or more physiological signals generated by one or more sensors. The apparatus may be configured to detect, from the one or more physiological signals, seed events suggestive of sleep disordering events. The apparatus may be configured to compute features indicative of patterns within portions of the one or more physiological signals that are associated with the detected seed events. The apparatus may be configured to apply to a classifier, the computed features indicative of patterns of the seed events. The classifier may be trained to compute a degree of fit of the computed features to learned repetitive patterns of sleep disordering events. The apparatus may be configured to output an identification of sleep disordering event(s) corresponding with the seed events based on the computed degree of fit determined by the classifier.

### 1 CROSS REFERENCE TO RELATED APPLICATIONS

This application claims the benefit of European Patent Application No. 21173562.6 filed 12 May 2021, the disclosure of which is hereby incorporated herein by reference.

### 2 BACKGROUND OF THE TECHNOLOGY

### Field of the Technology

The present technology generally relates to one or more of the screening, detection, diagnosis, monitoring, treatment, prevention and amelioration of disorders such as respiratory-related disorders. The present technology also relates to medical devices or apparatus, and their use. In some implementations, technology involves the screening, detection diagnosis, monitoring, treatment, prevention and/or amelioration of sleep disordering events such as sleep disordered breathing, including for example, central or obstructive apnea.

### Description of Related Art

### Therapies

A range of therapies have been used to treat or ameliorate respiratory conditions. Furthermore, otherwise healthy individuals may take advantage of such therapies to prevent respiratory disorders from arising.

Nasal Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion by pushing the soft palate and tongue forward and away from the posterior oropharyngeal wall.

High flow therapy (HFT) does not rely on positive airway pressure within a sealed patient interface, but rather on delivery of air at a therapeutic flow rate to the vicinity of the entrance(s) to the patient's airway via an unsealed patient interface that may be significantly open to atmosphere. High flow therapy has been used to treat OSA, CSR, and COPD. The air being delivered to the airway at a high flow rate, relative to typical respiratory flow rates, flushes out the patient's anatomical deadspace and decreases the amount of rebreathed CO₂, thereby increasing the efficiency of gas exchange. High flow therapy may be used in conjunction with respiratory pressure therapy.

Non-invasive ventilation (NIV) therapy provides ventilatory support to a patient through the upper airways to assist the patient in taking a full breath and/or maintaining adequate oxygen levels in the body by doing some or all of the work of breathing. NIV is provided via a non-invasive patient interface. NIV has been used to treat CSR, OHS, COPD, NMD, and Chest Wall disorders.

### Diagnosis and Treatment Systems

These therapies may be provided by a treatment system or device. Such systems and devices may also be used to diagnose a condition without treating it.

A treatment system may comprise a Respiratory Therapy Device (RT device) such as Respiratory Flow Therapy Device or a Respiratory Pressure Therapy Device (RPT device), such as a high flow therapy device (HFT device), an air circuit, a humidifier, and a patient interface.

### Patient Interface

A patient interface may be used to interface respiratory equipment to its user, for example by providing a flow of breathable gas. The flow of breathable gas may be provided via a mask to the nose and/or mouth, a tube to the mouth or a tracheostomy tube to the trachea of the user. Depending upon the therapy to be applied, the patient interface may form a seal, e.g., with a face region of the patient, to facilitate the delivery of gas at a pressure at sufficient variance with ambient pressure to effect therapy, e.g., a positive pressure of about 10 cmH₂O. For other forms of therapy, such as the delivery of oxygen, the patient interface may not include a seal sufficient to facilitate delivery to the airways of a supply of air at a positive pressure of about 10 cmH₂O.

### Respiratory Therapy (RT) Devices

Air pressure generators are known in a range of applications, e.g., industrial-scale ventilation systems. However, air pressure generators for medical applications have particular requirements not fulfilled by more generalised air pressure generators, such as the reliability, size and weight requirements of medical devices. In addition, even devices designed for medical treatment may suffer from shortcomings, including one or more of comfort, noise, ease of use, efficacy, size, weight, manufacturability, cost, and reliability.

One known RPT device used for treating sleep disordered breathing is the S9 Sleep Therapy System, manufactured by ResMed, which proves CPAP therapy. Another example of an RPT device is a ventilator. Ventilators such as the ResMed Stellar™ Series of Adult and Paediatric Ventilators may provide invasive and non-invasive non-dependent ventilation therapy for a range of patients for treating a number of conditions such as but not limited to NMD, OHS and COPD.

RPT devices typically comprise a pressure generator, such as a motor-driven blower or a compressed gas reservoir, and are configured to supply pressurised air to the airways of a patient. The outlet of the RPT device is connected via an air circuit to a patient interface such as those described above.

There may still be a need for a system that can not only monitor the health of the user, but can also help those users understand how to improve their own quality of life, such as by understanding their symptoms and triggers, and how they respond to medication.

Additionally, it is desirable for the system to have access to, and be capable of processing, as much data relevant to the user's condition as possible. In this respect, it is also desirable that the system be capable of monitoring the patient for as much of the day and night as possible, not just when the patient is in bed.

### Screening/Diagnosis/Monitoring Systems

Screening and diagnosis generally describe the identification of a disorder from its signs and symptoms. Screening typically gives a true/false result such as indicating whether or not a patient's disorder is severe enough to warrant further investigation, while diagnosis may result in clinically actionable information. Screening and diagnosis tend to be one-off processes, whereas monitoring the progress of a disorder can continue indefinitely. Some screening/diagnosis systems are suitable only for screening/diagnosis, whereas some may also be used for monitoring.

Polysomnography (PSG) is a conventional system for diagnosis/monitoring of cardio-pulmonary disorders, and typically involves expert clinical staff to apply the system. PSG typically involves the placement of 15 to 20 contact sensors on a person in order to record various biosignals such as electroencephalography (EEG), electrocardiography (ECG), electrooculograpy (EOG), electromyography (EMG), etc. PSG for sleep disordered breathing has involved two nights of observation of a patient in a clinic, one night of pure diagnosis and a second night of titration of treatment parameters by a clinician. Clinical experts may be able to diagnose or monitor patients adequately based on visual observation of PSG signals. However, there are circumstances where a clinical expert may not be available, or a clinical expert may not be affordable. PSG is therefore expensive and inconvenient. In particular it is unsuitable for in-home diagnosis/monitoring.

In the field of sleep analysis, the scoring or detection of sleep disordering events, such as obstructive apnea, central apnea, obstructive hypopnea, central hypopnea, or respiratory effort related arousals (RERAs) is generally effected by such polysomnography during which different body functions are monitored such as the brain by electroencephalography (EEG), the eye movements by electrooculography (EOG), the muscle activity or skeletal muscle activation by electromyography (EMG), heart rhythm by electrocardiography (ECG), and respiratory airflow. Despite it being considered as the gold standard, this technique has several drawbacks. First, the test itself may require hospitalization or set-up in the home environment by a healthcare professional. Second, the interpretation of the test is often not fully automated, requiring a sleep technician to manually analyse the recorded signals thereby introducing inter-scorer variation. Third, the test may interfere with the person's sleep due to the complex wiring and overall overhead thereby influencing clinical parameters such as supine sleep time, sleep onset, arousals during sleep, and so forth. Fourth, sleeping disorders such as sleep apnea are known to have a high inter-night variability, and the current diagnostic systems are not suited for a multi-night study due the clinical shortcomings, lack of convenience, and high cost per examination.

Another convenient screening/diagnosis/monitoring system for home use comprises a nasal cannula, a pressure sensor, a processing device, and recording means. A nasal cannula is a device comprising two hollow open-ended projections that are configured to be inserted non-invasively a little way into a patient's nares so as to interfere as little as possible with the patient's respiration. The hollow projections are in fluid communication with a pressure transducer via a Y-shaped tube. The pressure transducer provides a data signal representative of the pressure at the entrance to the patient's nares (the nasal pressure). It has been shown that a nasal pressure signal is a satisfactory proxy for the nasal flow rate signal generated by a flow rate transducer in-line with a sealed nasal mask, in that the nasal pressure signal is comparable in shape to the nasal flow rate signal. The processing device may be configured to analyse the nasal pressure signal from the pressure transducer in real time or near real time to detect and classify SDB events in order to monitor the patient's condition. Screening or diagnosis may require similar analysis but not necessarily in real time or near real time. The recording means is therefore configured to record the nasal pressure signal from the pressure transducer for later off-line or “batch” analysis by the processing device for screening/diagnosis purposes.

More recently, the scoring of sleep events has also been performed using a different technique based on measurements by a photoplethysmogram (PPG). PPG measurements at one or more wavelengths allow the monitoring of changes in the peripheral arterial tone, which is indicative of peripheral vasodilation or vasoconstriction, allow deriving the oxygen saturation (SpO₂), and allow deriving the pulse rate. An advantageous embodiment of a system allowing deriving occurrences of sleep events from changes in the peripheral arterial tone (PAT) was for example disclosed in EP 3593707 A1. This system, as well as the measurement technique used in the system, allow the detection of sleep disordering events with a minimal impact on, and disturbance of, the sleeping subject, which is a considerable advantage over polysomnography.

Both in polysomnography as well as in a PAT-based approach, the scoring of sleep disordering events is generally based on a co-occurrence of a number of physiological phenomena. In polysomnography, an event may be qualified as a respiratory related sleep disordering event, also called respiratory event, when one or more phenomena co-occur among for example the following: a reduction or even an absence of airflow, an arousal which may be detected by EEG, an oxygen desaturation, detected by a drop in an SpO₂ graph, followed by a return to baseline. For each of these phenomena, thresholds have been determined, for example by the American Academy of Sleep Medicine (AASM), above or below which a measurement may be qualified as being indicative of a respiratory event. In a PAT-based approach as well, co-occurrence of an oxygen desaturation, an increase in heart rate and/or an increase in PAT is considered as being indicative of a respiratory event.

A problem linked with the present approach is that some sleep disordering events may be missed in the scoring process, for example when one of the measurements does not reach a predefined threshold for that feature. Similarly, the scoring process may lead to false positive detections of sleep disordering events. Moreover, home sleep testing devices, such as PAT-based devices or cardiorespiratory polygraphy devices, do not exclusively make use of the gold-standard signals derived from polysomnography, such as airflow or EEG, and therefore, are at an implicit disadvantage in detecting sleep disordering events as they have to infer respiratory events from alternative signal modalities.

Improvements are needed for providing a cost effective or more readily accessible screening or monitoring tool such as to assist in raising awareness of SDB conditions and/or to promote treatment of SDB. The realization of such a reliable and cost-effective screening apparatus such as for home use remains a considerable technical challenge. It is therefore an aim of some versions of the present technology to solve or at least alleviate one or more of the above-mentioned problems. Thus, the technology aims at providing an improved methods and apparatus for detecting sleep disordering events with a relatively high accuracy.

### 3 BRIEF SUMMARY OF THE TECHNOLOGY

The present technology is directed towards providing medical devices used in the screening, monitoring, diagnosis, amelioration, treatment, or prevention of respiratory disorders having one or more of improved comfort, cost, efficacy, ease of use and manufacturability.

Some implementations of the present technology may include a processor-implemented method for detecting sleep disordering events. The method may include accessing a plurality of physiological signals generated by one or more sensors. The method may include detecting, from the plurality of physiological signals, seed events suggestive of sleep disordering events. The method may include computing features indicative of patterns within portions of the plurality of physiological signals that are associated with the detected seed events. The method may include applying, to a classifier, the computed features indicative of patterns of the seed events. The classifier may be trained, such as by or with a machine learning classification algorithm, to compute a degree of fit of the computed features to learned patterns of sleep disordering events. The method may include outputting an identification of one or more sleep disordering events corresponding with the seed events based on the computed degree of fit determined by the classifier.

In some implementations, the classifier may be one or more of a machine learning classifier, a decision tree model, a machine learning classifier model, a logistic regression classifier model, a neural network, Naive Bayes classifier model, and a support vector machine. The plurality of physiological signals may include a peripheral arterial tone (PAT) signal, and one or more of: an oxygen saturation signal, a pulse rate signal, a respiratory effort signal, a movement signal, and an air flow signal (e.g., flow rate signal). In some implementations, each seed event of the seed events may include one or more of: an amplitude drop from a baseline within a peripheral arterial tone (PAT) signal, a desaturation of oxygen in an oxygen saturation (SpO2) signal, an amplitude increase from a baseline in a pulse rate (PR) signal, an amplitude change (such as an increase or decrease) from a baseline in a respiratory effort signal, and an amplitude change (such as an increase or decrease) from a baseline in air flow rate signal. The patterns may include a morphological pattern. The patterns may include a temporal pattern.

In some implementations, a feature of the computed features may include one or more of: a duration of a seed event; an intensity of a seed event; a derived inclination or slope of a seed event; a derived morphological asymmetry of changing slope of a vicinity of a seed event; a depth of a seed event; a variance of the signal amplitudes of the seed event; an average of the signal amplitudes of the seed event; a skewness of the seed event; and a characterization of a morphological shape of a seed event. One or more features of the computed features may include one or more of a determined starting point of the seed event, a determined end point of the seed event, a determined point of highest or lowest intensity, and a determined characteristic point of a seed event. The detected seed events may include a first seed event of a first signal of the plurality of physiological signals and a second seed event of a second signal of the plurality of physiological signals. The second signal may be a different physiological signal from the first signal, and one or more features of the computed features may characterize the first seed event and the second seed event. For example, one or more features of the computed features may characterize the first seed event in relation to the second seed event. Such a characterization may, for example, serve to associate one seed event from one physiological signal with another seed event from a different physiological signal, such as an alignment of one seed event from one physiological signal with another seed event from a different physiological signal. The one or more features of the computed features that characterize the first seed event in relation to the second seed event may include: (a) a time amount that a desaturation nadir trails or precedes a peak pulse rate increase and/or a PAT signal amplitude decrease; and/or (b) a timing difference between a detected pulse rate (PR) surge peak and a decrease valley of a PAT signal.

The detected seed events may include a third seed event of the first signal, and the one or more features of the computed features may characterize the first seed event and the third seed event. For example, the one or more features of the computed features may characterize the first seed event in relation to the third seed event. Such a characterization may, for example, serve to associate seed events from one physiological signal. Optionally, the first seed event and the third seed event may be a neighboring pair of seed events. In some implementations, the one or more features of the computed features that characterizes the first seed event and the third seed event may include any one or more of: (a) a duration between the first seed event and the third seed event; (b) a computed stability of a period between the first seed event and the third seed event; and (c) a computed stability of the first seed event and the third seed event. The computing of the duration may include detection of a characteristic point in each of the first seed event and the third seed event and determining the duration based on an interval associated with the detected characteristic points. The detected characteristic points may include one or more of a local amplitude minimum and local amplitude maximum. The computed stability may be derived from a plurality of seed events and may include one or more of a depth, average, and a variance, and/or may be, for example, a stability of depths of the events.

In some implementations, the detected seed events may include a fourth seed event of the second signal, wherein one or more features of the computed features may characterize (a) the first seed event and the third seed event of the first signal and (b) the second seed event and the fourth seed event of the second signal. For example, the one or more features of the computed features may characterize (a) the first seed event and the third seed event of the first signal in relation to (b) the second seed event and the fourth seed event of the second signal. Such characterizations may, for example, serve to associate seed events of one physiological signal with seed events of another different physiological signal, such as to detect overlap of sequences of seed events from different physiological signals. For example, the one or more features of the computed features that characterizes (a) the first seed event and the third seed event of the first signal and (b) the second seed event and the fourth seed event of the second signal may include: a temporal correspondence of (a) detected pulse rate (PR) peaks of seed events of a PR signal, and (b) reduction in peripheral arterial tone (PAT) to a minimum point in valleys of seed events of a PAT signal.

In some implementations, the method may further include generating the outputting of the identification as feedback in response to a user input of a selection, on user interface, of at least one seed event detected by the detecting implemented by one or more processors. The method may further include generating a signal for controlling operation of a respiratory therapy apparatus based on the outputting or the applying. The generating may include transmitting the identification of the one or more sleep disordering events to a remote computing system or server. The generating may include transmitting the signal to the respiratory therapy apparatus via a network communications link.

Some implementations of the present technology may include a controller that may include at least one processor and at least one memory including processor control instructions. The at least one memory and processor control instructions may be configured to, with the at least one processor, cause the controller to perform a method including any one or more aspects of the methodologies as describe herein.

Some implementations of the present technology may include apparatus for detecting sleep disordering events. The apparatus may include one or more sensors. The apparatus may include a controller. The controller may include one or more processors and at least one memory including processor control instructions. The controller may be configured to access a plurality of physiological signals generated by one or more sensors. The controller may be configured to detect, from the plurality of physiological signals, seed events suggestive of sleep disordering events. The controller may be configured to compute features indicative of patterns within portions of the plurality of physiological signals that are associated with the detected seed events. The controller may be configured to apply, to a classifier, the computed features indicative of patterns of the seed events. The classifier may be trained to compute a degree of fit of the computed features to learned patterns of sleep disordering events. The controller may be configured to output an identification of one or more sleep disordering events corresponding with the seed events based on the computed degree of fit determined by the classifier.

Some implementations of the present technology may include a processor-readable storage medium that may include processor-executable instructions for performing a method including any one or more aspects of the methodologies as describe herein when executed by one or more processors.

Some implementations of the present technology may include a processor-readable medium, having stored thereon processor-executable instructions which, when executed by one or more processors, cause the one or more processors to detect sleep disordering events. The processor-executable instructions may include instructions to access a plurality of physiological signals generated by one or more sensors. The processor-executable instructions may include instructions to detect, from the plurality of physiological signals, seed events suggestive of sleep disordering events. The processor-executable instructions may include instructions to compute features indicative of patterns within portions of the plurality of physiological signals that are associated with the detected seed events. The processor-executable instructions may include instructions to apply, to a classifier, the computed features indicative of patterns of the seed events, wherein the classifier is trained to compute a degree of fit of the computed features to learned patterns of sleep disordering events. The processor-executable instructions may include instructions to output an identification of one or more sleep disordering events corresponding with the seed events based on the computed degree of fit determined by the classifier.

In some implementations, the processor-executable instructions may further include instructions to generate a signal for controlling operation of a respiratory therapy apparatus based on the outputting or applying. The controlling operation may include controlling a pressure or flow therapy of a blower of the respiratory therapy apparatus.

Some implementations of the present technology may include a server with access to any of the processor-readable medium described herein. The server may be configured to receive requests for downloading the processor-executable instructions of the processor-readable medium to a processing device over a network.

Some implementations of the present technology may include a processing device. The processing device may include: one or more processors; and (a) any processor-readable medium described herein, or (b) wherein the processing device is configured to access the processor-executable instructions with any server described herein. The processing device may be a respiratory therapy apparatus. The processing device may be configured to generate a pressure therapy or a flow therapy.

Some implementations of the present technology may include a method of a server having access to any processor-readable medium described herein. The method of the server may include receiving, at the server, a request for downloading the processor-executable instructions of the processor-readable medium to an electronic processing device over a network. The method of the server may include transmitting the processor-executable instructions to the electronic processing device in response to the request.

Some implementations of the present technology may include a method of one or more processors for detecting sleep disordering breathing events. The method may include accessing, with the one or more processors, any processor-readable medium described herein. The method may include executing, in the one or more processors, the processor-executable instructions of the processor-readable medium.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### BRIEF DESCRIPTION OF THE DRAWINGS

The present technology is illustrated by way of example, and not by way of limitation, in the figures of the accompanying drawings, in which like reference numerals refer to similar elements including:

FIG. 1 shows a first schematic graph representing of a plurality of physiological signals over time.

FIG. 2 shows a second schematic graph representing of a plurality of physiological signals over time.

FIG. 3 shows the second schematic graph of FIG. 2 illustrating an embodiment of the method according to the present technology.

FIG. 4 shows a third schematic graph representing of a plurality of physiological signals over time.

FIG. 5 shows a fourth schematic graph representing of a plurality of physiological signals over time.

FIG. 6A shows an example finger sensor apparatus on patient suitable for implementations of the present technology.

FIG. 6B shows an example computing system or processing device suitable for performing various steps according to example implementations of the present technology, such as with the sensor apparatus of FIG. 6A.

FIG. 7 is an example process of one or more processors, such as in a system of the present technology, with a methodology for detecting sleep disordering events of the present technology.

FIG. 8 is another example process for one or more processors, such as in a system of the present technology, for methodology detecting sleep disordering events of the present technology.

FIG. 8A shows an example system in accordance with the present technology. A patient 1000 wearing a patient interface 3000 receives a supply of pressurised air from an RPT device 4000. Air from the RPT device 4000 is humidified in a humidifier 5000, and passes along an air circuit 4170 to the patient 1000. A bed partner 1100 is also shown.

FIG. 8B shows an RPT device 4000 in use on a patient 1000 with a nasal mask 3000.

FIG. 8C shows an RPT device 4000 in use on a patient 1000 with a full-face mask 3000.

FIG. 9 shows an example non-invasive patient interface 3000 in the form of a nasal mask.

FIG. 10A shows an RPT device 4000 in accordance with one form of the present technology.

FIG. 10B shows a schematic diagram of the pneumatic circuit of an RPT device 4000 in accordance with one form of the present technology. The directions of upstream and downstream are indicated.

FIG. 10C shows a schematic diagram of the electrical components of an RPT device 4000 in accordance with one aspect of the present technology.

FIG. 10D shows a schematic diagram of the algorithms 4300 implemented in an RPT device 4000 in accordance with an aspect of the present technology. In FIG. 10D, arrows with solid lines indicate an actual flow of information, for example via an electronic signal.

FIG. 10E is a flow chart illustrating a method 4500 carried out by the therapy engine module 4320 of FIG. 10D in accordance with one aspect of the present technology.

FIG. 11 shows a humidifier 5000.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

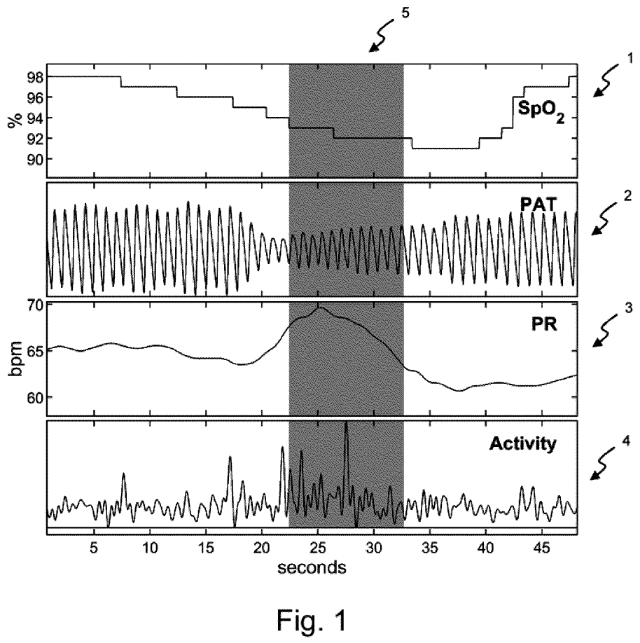

In example implementations, a processor-implemented or computer-implemented process detects sleep disordering events. The method may include obtaining at least one physiological signal, which signal may include a pointer to a sleep disordering event as is known to the person skilled in the art, identifying seed events suggestive of sleep disordering events from the at least one physiological signal, determining patterns, within at least part of the seed events, determining degrees of fit of the seed events to the patterns, and detecting sleep disordering events based on the determined degrees of fit. Aspects of such a methodology may be considered in relation to the graphs of FIGS. 1 through 5.

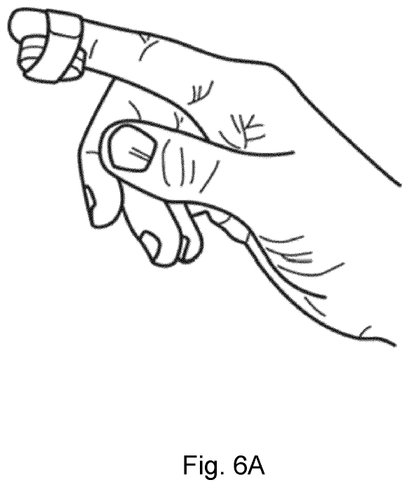

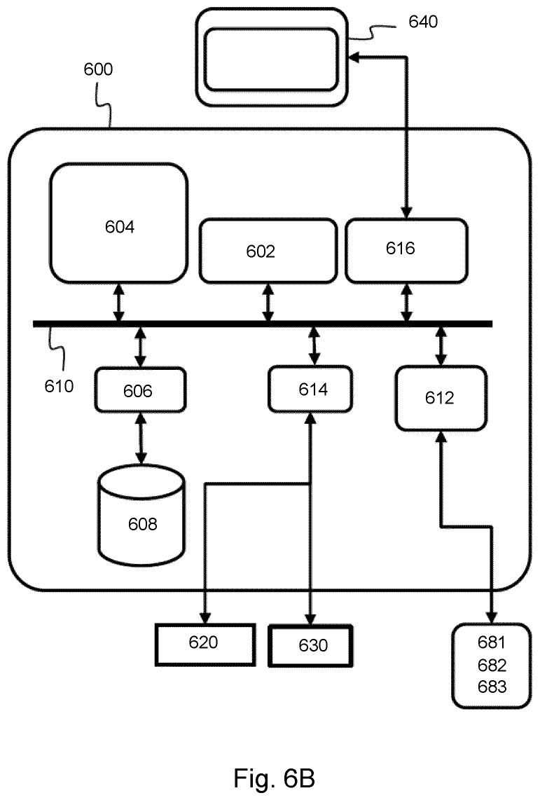

For example, FIG. 1 shows a first schematic graph representing an example plurality of physiological signals over time, any one or more of which may be utilized in the present technology. In the present graph, the physiological signals include a saturation of oxygen (SpO₂) signal 1, a PAT signal 2, a pulse rate signal 3 and a limb activity signal 4. A PAT-signal or PAT measurement is a measurement of changes in the pulsatile arterial blood volume which reflect changes in arterial tone, and which can for example be measured by mounting a pneumatic or optical sensor at a fingertip of a patient. A pulse rate signal may be obtained in many different ways known to the person skilled in the art. Oxygen saturation (SpO₂) can be measured by a pulse oximeter, which may be attached to, for example, a patient's finger, nostril, wrist or earlobe. The saturation of oxygen, PAT and pulse rate signals 1, 2, 3 may for example be derived from measurements by a single device, for example by measuring arterial pulsatile volume changes at the fingertip, for example by a device as disclosed in EP3,593,707 or U.S. Patent Application Publication No. 2020/0015737, the entire disclosure of which is incorporated herein by reference. Such a device may be configured to perform photoplethysmography (PPG), preferably dual wavelength photoplethysmography, from which a PAT signal, an SpO₂ signal and a pulse rate signal can be derived. An example of such a finger sensor apparatus is illustrated in FIG. 6A. However, the measurements may also be measured by separate devices, such as any of the devices described herein.

The activity signal 4 may, for example, be derived from a movement sensor, such as an accelerometer attached to an arm or a leg of a patient or a noncontact sensor, such as a RF motion sensor or a sonar based sensor. For example, a radio frequency (RF) transceiver of an RF sensor may serve as a noncontact part of the sensing apparatus. Such an RF sensing device, which may be integrated with or coupled to the processing apparatus described herein, and may be implemented with any of the techniques and sensor components described in U.S. Patent Application Publication No. 2014/0024917, entitled “Range Gated Radio Frequency Physiology Sensor” and filed on Jul. 18, 2013; International Patent Application No. PCT/EP2017/070773, entitled “Digital Radio Frequency Motion Detection Sensor” and filed on Aug. 16, 2017; and U.S. Patent Application Publication No. 2018/0239014, entitled “Digital Range Gated Radio Frequency Sensor” and filed on Aug. 16, 2016, the entire disclosure of each of which are incorporate herein by reference. A sonar based sensor may be, for example, a microphone and speaker implemented processing device such as any of the processing devices described in U.S. Patent Application Publication No. 2021/0275056 filed on 19 Sep. 2017 or U.S. Patent Application Publication No. 2022/0007965, filed on 19 Nov. 2019, the entire disclosures of which are incorporated herein by reference. Limb activity can be derived from, among others, an accelerometer, for example attached to an arm or a leg of a patient. Devices which are configured to be attached to a fingertip or a wrist may be preferred in sleep analysis over devices which are attached to the head or chest because the latter devices may hinder natural sleep and thus influence results. Other sensors and physiological signals may also be implemented.

The choice for a given combination of physiological signals may, for example, be determined by the type of sleep disordering events that need to be distinguished, or by a potential influence of the obtaining of the signal on one's sleep. Many more combinations of physiological signals are possible, as will be clear to the person skilled in the art. For example, any of a respiratory effort signal and an airflow signal may also be implemented. Respiratory effort can be derived from, among others, an accelerometer, a bioimpedance measurement, or an inductance plethysmography belt. Airflow can be derived from a measurement by a pressure sensor, flow rate sensor or an air temperature sensor which may be mounted to a patient's nostril, which is also known to the person skilled in the art. In general, such signals are obtained in a common time span. Such common time span does not mean that the measurements of a plurality of physiological signals all need to have a same duration or all need to start and stop at the same moment. By ‘common time span’, it is meant that the physiological signals need to at least partly overlap in time such that they include a time span which is in common or the same for a plurality of said signals and such that said common time span includes seed events.

In prior art methods, an amplitude of a drop in SpO₂ as observed in the saturation of oxygen signal 1 would mainly be compared to a threshold value, as set for example by the AASM, to decide whether or not the drop in SpO₂ could be assessed as a sleep disordering event. A similar way of proceeding is used for the drop in PAT amplitude in the PAT signal 2 or for the pulse rate and activity spike in the respective signals 3 and 4. If all or a number of these four indicators point to a potential sleep disordering event, the prior art method would come to the conclusion that a single apneic or sleep disordering event has been detected. In these prior art methods for detecting sleep disordering events, potential indicators of sleep disordering events in physiological signals are mainly treated individually, i.e. compared individually to threshold values without taking into account any context information from the rest of the physiological signal. In this regard, at most, prior art algorithms combine threshold values of more or less coincidental indicators, as shown in FIG. 1, where a co-occurrence 5 of an SpO₂ drop below a predefined threshold, a PAT amplitude drop below a predefined threshold, a pulse rate spike above a predefined threshold and a limb activity spike above a predefined threshold is observed.

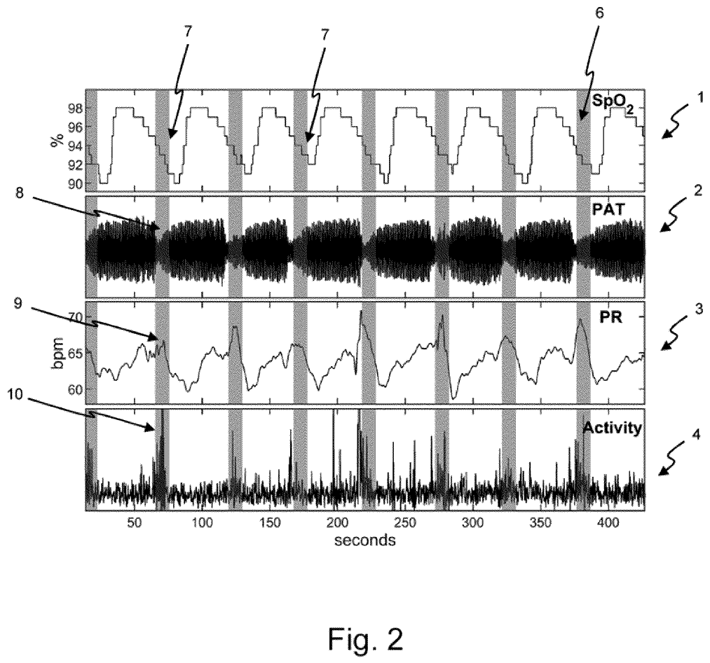

Unlike the prior methodologies, implementations of the present technology consider a greater context of such signals, such as with pattern recognition, for identifying events, within a given signal and/or across multiple signals. For example, FIG. 2 shows a second schematic graph representing of a plurality of physiological signals spanning a common time span. As in the first graph, the physiological signals include a saturation of oxygen (SpO₂) signal 1, a PAT signal 2, a pulse rate signal 3 and a limb activity signal 4. In a method, such as one implemented by one or more processors, according to the present technology, any one or more of these physiological signals or the aforementioned physiological signals, for example only a SpO₂ signal 1 or any combination of at least two or more of these signals, may be evaluated for detecting sleep disordering events. According to the method, seed events 6, which are suggestive of sleep disordering events, are selected. The seed events 6 may, for example, be detections of one or more of a desaturation of oxygen 7, a PAT signal amplitude drop 8 from a baseline, a pulse rate signal amplitude increase 9 from a baseline, a respiratory effort signal amplitude decrease from a baseline, an increase in limb activity 10 or any other known physiological signal which may be a marker of a sleep disordering event. The seed events 6 are only suggestive of a sleep disordering event since they do not necessarily point to a sleep disordering event since they may have another underlying cause.

Thus, the seed events may for example be at least one of a PAT signal amplitude drop from a baseline, a desaturation of oxygen, a pulse rate signal amplitude increase from a baseline, a respiratory effort signal amplitude decrease from a baseline, an airflow signal amplitude decrease from a baseline, and a limb activity increase from a baseline. These seed events are suggestive of a sleep disordering event, meaning that they may be a sign or symptom of a sleep disordering event, but are not necessarily associated with a sleep disordering event. In other words, the seed events are events which may, but will not necessarily, be assessed as associated with sleep disordering events. When other physiological signals are obtained, seed events can be defined differently depending on the signal.

Optionally, a seed event from one physiological signal may be generally associated with a similarly timed seed event from other physiological signal(s) so that a set of associated seed events (e.g., one from each signal) within a common time frame of a plurality of physiological signals may be taken as suggestive of a candidate respiratory event. An example of a set associated seed events of common time scale may be a desaturation seed event of a PPG signal that temporally coincides with a PAT signal amplitude reduction, a pulse rate signal increase, and an increase in an activity count of a movement signal.

It has been observed that sleep disordering events, which may be respiratory related sleep disordering events such as apnea events, snoring, RERA's, or non-respiratory related sleep disordering events, such as for example spontaneous autonomic arousals, generally occur in groups or sequences at a relatively constant interval. This contextual information may be exploited with the present technology. By qualifying events as sleep disordering events at least partly based on degrees of fit to patterns which are recognized in the data, the accuracy of the detection of sleep disordering events can be increased, since the scoring becomes less dependent on simple predetermined thresholds of prior art scoring. In the prior art, sleep disordering events have generally been analysed on a single-event or temporally isolated basis, determining whether or not an individual measurement or two or more co-occurring events in one or more physiological signals were above or below a predetermined threshold to qualify an event as a sleep disordering event without looking at the context of the event. As a result of such a single-event approach, sleep disordering events may have been missed, for example when at least one of the seed events associated with said sleep disordering events was for example just below or above such a threshold. When qualifying the seed events as being associated with sleep disordering or not, the present method, unlike the prior art, does more than simply compare each seed event to a predefined threshold. The present method may further determine patterns in the measured data taking into account an entire data set and then integrate this pattern into the qualification of the seed events as being associated with sleep disordering events or not, which allows the present technology to reach a better accuracy in the detection of sleep disordering events than prior art methods.

Accordingly, in a next step of the process, and unlike to prior art methods, patterns, such as repeated patterns or substantially regular patterns, will be determined within at least part(s) of each signal that contains the seed event(s) 6 that were selected in a physiological signal. That is, the particular part (e.g., samples) of the signal representing a vicinity of each seed event (e.g., samples of a signal representing a period of a desaturation from its beginning to its end) is evaluated further, such as by determining or computing one or more features with the particular part, such as for evaluating whether one or more patterns is present and/or is shared by multiple seed events. These patterns may include morphological patterns, i.e. related to a shape, of the representation of the physiological signal, and may be related to an amplitude of the signal corresponding to an intensity of a seed event, or an inclination, skewness or a morphological asymmetry of changing slope (e.g., an increase or decrease) of a vicinity of a seed event in the signal.

For example, such seed-based determination of morphological features for the SpO2 signal may, for example, include, among others, the length of a desaturation, the depth of the desaturation, and the slope asymmetry between the desaturation and its resaturation phase. Seed-based features concerning signal morphology may also be determined or computed from the seed event associated portions of the other physiological signal(s) as well. The determination of morphological patterns can thus include a determination of features that provide a characterization of a morphological shape of a seed event, such as using signal processing and characteristic signal data samples and/or statistical functions that provide a numeric value(s) that serve as a characterization of the shape (e.g., of the signal over time) of the vicinity of each seed event. Additionally, and/or alternatively, the process of determining or computing the aforementioned patterns may also optionally involve computing or determining a point of highest or lowest intensity and/or determining any other characteristic point of a seed event, which may serve as feature(s) for characterizing the seed event.

Thus, the patterns can include a morphological pattern. A morphological pattern may for example be linked to typical shape features of a representation of a seed event in the obtained signal. As an example, an SpO₂ decrease can show a relatively slow fall and a relatively steep rise to normal. The determining of such patterns can for example include at least one of determining a duration of a seed event, determining an intensity of a seed event, determining a morphological asymmetry of a seed event and/or characterizing a morphological shape of a seed event. A template morphology may for example be derived from a plurality of seed events, neighbouring in time or spaced apart in time. Shape features of a representation of a seed event may then be compared to said template. The determining of the patterns can further include determining features, such as those previously described, of the patterns, for example shape features, which can characterize the patterns, for example a shape of said patterns, such as for example an average morphological asymmetry or an average magnitude of the seed events within the substantially regular patterns.

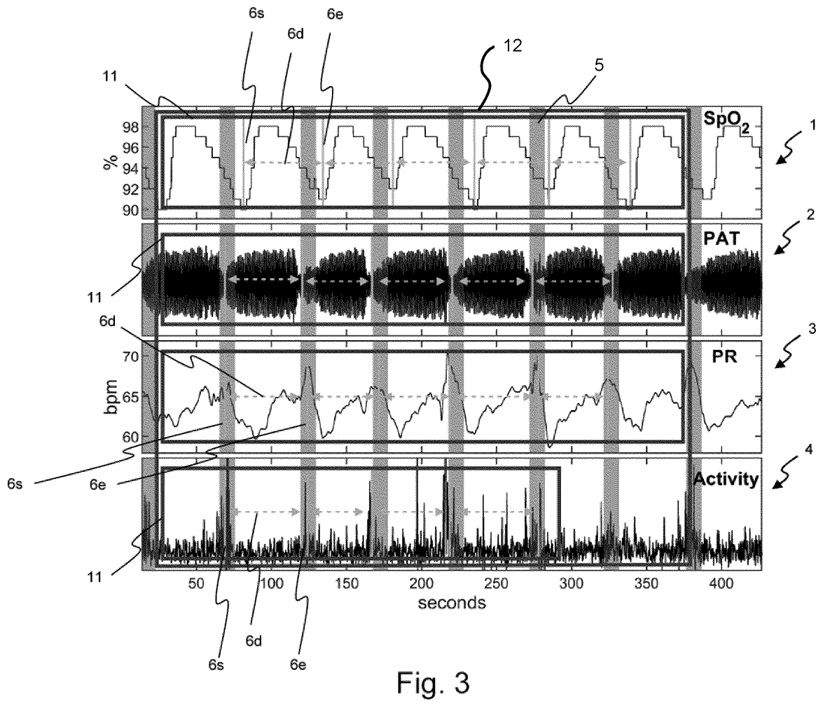

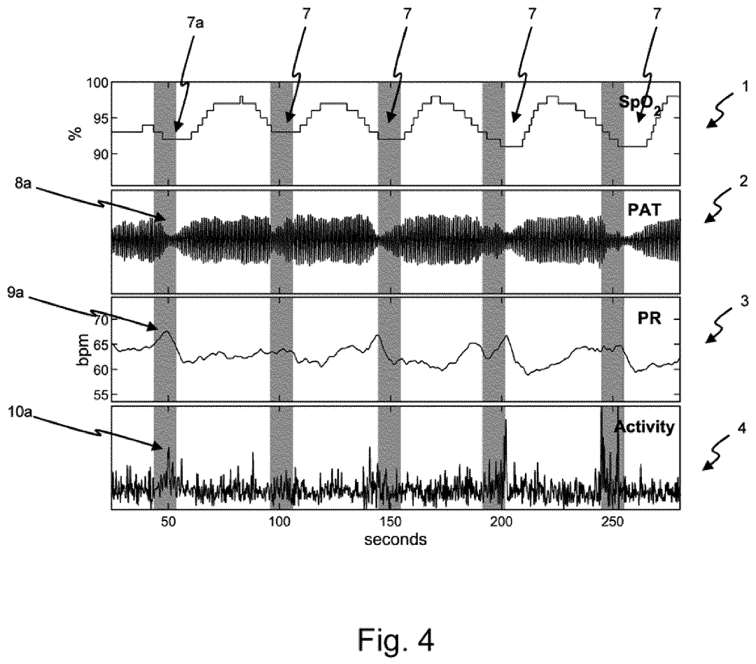

Furthermore, the patterns may also include temporal patterns, such as will be described in relation to FIG. 3 or FIG. 4. Such temporally related patterns can be especially well recognized, for example, in the SpO₂ signal 1 and in the PAT signal 2 but are also present in the other physiological signals. Thus, the process may further include a seed-based determination of temporal features concerning the particular part (e.g., samples) of the signal representing a vicinity of each seed event, such as using signal processing and characteristic signal data samples and/or statistical functions that provide a numeric value(s) that serve as a characterization of the temporal pattern of the characteristics of the vicinity of each seed event.

Thus, the patterns can include a temporal pattern, such as a repetition of a seed event at predetermined intervals. The determining of such patterns can therefore include at least one of determining a starting point of the seed event, determining an end point of the seed event, determining a point of highest or lowest intensity and/or determining any other characteristic point of a seed event. In this way, a temporal pattern can be recognized in a repetition of seed events, for example in a time difference between starting points of seed events, or between end points of seed events. A temporal distance or time difference between local maxima or minima in the obtained signal may also show a substantially regular temporal pattern. The deriving of the pattern can for example also include determining a feature comprising an interquartile range of a (normalized) difference between neighbouring seed events. The determining of the patterns can further include determining features of the pattern which can characterize the patterns, for example temporal features, such as for example a number of events in a repeated pattern. The determining of the patterns may further include determining a quality of the substantially regular patterns, for example by determining a stability or variability of periodicity between characteristic points of events represented by the substantially regular pattern, for example using a standard deviation or any other known parameter. Such temporal pattern features may be computed or determined so as to provide temporal sequencing information of, or between, seed events, such as neighbouring events, of the same signal. Furthermore, such temporal pattern(s) may be computed or determined so as to provide temporal alignment information of, or relative to, seed events across different physiological signals.

For example, with a group of seed events in a common time frame (e.g., one from each signal), the process may determine relative juxtaposition so as to characterize a temporal alignment of signal features of seed events from multiple different physiological signals. For example, the process may determine how much time (e.g., how many seconds) the SpO₂ signal desaturation nadir trails or precedes the peak of the pulse rate (PR) signal increase and PAT signal amplitude decrease. The degree of temporal alignment between the pulse rate (PR) surge peak and PAT decrease valley are also significant temporal characteristics as a perfect alignment is a significant indicator of an autonomic arousal, which is a sympathetic activation that often occurs near the end stage of a respiratory event.

As a further example, two seed events from different signals may be considered in a temporally aligned pattern when the feature of the signals include, for example, a time feature of a highest point of a pulse rate spike of a seed event that coincides with a time feature of a lowest PAT signal amplitude of another seed event, when both events are preceded by a gradual decrease in peripheral oxygen saturation and followed by a lowest value in oxygen saturation and a steep rise of oxygen saturation. Time differences between onsets of seed events in different signals may be found to be relatively constant. An order between seed events may slightly vary from person to person but may remain relatively constant for a given patient. Other features may intervene in the step of grouping seed events from the plurality of physiological signals into sets, such as for example a substantial alignment in time of local minima or maxima of physiological signals, in particular a local minimum of oxygen desaturation preceding PAT signal and/or pulse rate local minima or maxima. Another example of a pattern in a co-occurrence of seed events may be a detection of a local minimum of a PAT signal and/or pulse rate towards an end of a flow reduction event.

By way of additional example, temporal (and/or morphological) patterns may be evaluated with regard to the seed events of the same physiological signal. Thus, the process may determine sequencing related features, such as with one or more pairs of neighboring seed events. For example, FIG. 3 shows the second schematic graph of FIG. 2 illustrating further aspects of a process of the present technology. In this regard, the process of determining repetitive patterns or temporal patterns, for example substantially regular temporal patterns, such as in relation to temporal sequencing of a seed event of one signal to another seed event (e.g., neighboring pairs) of the same signal, can for example include determining features comprising a duration or time span 6d between subsequent seed events 6, for example by determining a characteristic point 6s, such as a local minimum or maximum of the amplitude of the seed event 6, and determining a subsequent characteristic point 6e of the subsequent seed event. This process may be performed in one or more of the physiological signals separately. Such processing can assist when respiratory events, and by implication their associated seed events pair, tend to occur in sequences, such as with respect to repetitive seed events or repetition of their signal characteristics.

Additionally, to further characterize each such sequence (e.g., a portion of a physiological signal that begins and ends with a vicinity of a first seed event and a vicinity of a second seed event such as where the first and second seed events are neighboring seed events), for each such sequence associated with a plurality of the seed events (e.g., the neighboring pairs of a signal), the process may determine additional sequencing related features that may be considered in relation to the aforementioned pattern assessments. For example, the process may determine features to characterize the period of, or between, each of the neighboring pairs of seed events according to stability of this period. Such stability of the physiological signal, such as between a neighboring pair(s), of seed events may be characterized by, for example, one or more statistical functions such as, variance, etc. such as by determining variance or other stability computation concerning features of seed events such as depth of each, etc. Similarly, the process may further characterize the seed events within a sequence by other statistical function, such as by computing average event morphology. Additionally, the process may evaluate an average of the number of samples of the signal in between the vicinities of the seed events as a feature. Similarly, the process may characterize the stability of the seed events within a sequence, such as by computing stability of event morphology of the samples of the signal in the vicinities of the seed events. A highly repetitive (i.e., having a highly stable period) sequence of seed events of a very similar morphology may be taken as having a high likelihood of being associated with a sequence of respiratory events. Still further, a computed feature of a pattern concerning a sequence may be an interquartile range of a (normalized) difference between neighbouring seed events.

The evaluation of each such sequence of a signal may be extended with a determination or computing or determination of additional features (e.g., temporal features associated with a temporal pattern) that characterize the sequence relative to other sequences (e.g., a sequence associated with a plurality of seed events, e.g., a neighboring pair or multiple neighboring pairs of seed events, from other physiological signals). Such a process provides information to characterize a relationship of sequences of different physiological signals (e.g., time overlapping sequence). In this regard, a wealth of information is contained in the synchronization between such sequences of different seed event signal types. For example, a PR sequence for which all PR peaks perfectly align with the valleys of the PAT reduction events of a PAT sequence renders it very likely that all seed events in those sequences are manifestations of (a sequence of) respiratory events. Such features may include the timing of characteristic points of the seed events within different signals (e.g., the PR, PAT and activity sequences) such as to confirm temporal alignment. Similarly, feature(s) may characterize a delay in alignment of the sequence of seed events of the PPG signal relative to the sequence the other signals since desaturation nadir typically trails the timing of changes in the other signals in a very systematic way.

As an example, a plurality of seed events in the physiological signals 1, 2, 3 and 4 represented in FIG. 3 have been grouped or coupled within the respective physiological signal 1, 2, 3 and 4 based on a similar event duration 6d, as indicated by the coupling rectangles 11. These sequences of seed events or coupled seed events may be indicatory of a repetition of a sleep disordering event. Additionally, and/or alternatively, to the coupling of seed events 6 within a single physiological signal, the process further determines features to relate sequences of seed events from the plurality of different physiological signals into aligned/related sequence sets 12, the seed events grouped into the set 12 being indicative of the same sleep disordering event. In other words, the sets are groups of seed events of different physiological signals, and the sets include seed events spanning a same time span and can be indicative of a same sleep disordering event. Explained in a more visual way, the sets 12 are ‘vertical’ groups or related sequences of seed events from different signals, whereas the coupling of seed events as described before is done ‘horizontally’ which are related seed events of a single signal. The grouping of seed events into the set can include determining a substantially regular pattern in a co-occurrence of the seed events of the set. As an example, a median duration or an onset of a seed event may be similar across different physiological signals. In FIG. 3, such a set 12 of seed events has been indicated.

Thus, with any combination of the aforementioned determined/computed features (e.g., temporal and/or morphological features from the seed events indicative of the aforementioned patterns, from aligned/related seed events of different signals, from seed event sequences for each signal and/or from aligned/related sequences of the seed events from different signals) that are associated with at least one seed event, may then be combined (such as in a feature vector or other data structure of such features) and applied to a processing algorithm such as in one or more digital processor(s), such as a classifier (e.g., a trained model produced by a machine learning classifier such as a rules based model or a decision tree) or other machine learning algorithm. The classifier or machine learning classifier, such as a trained model from such a classifier, may be configured to determine degrees of fit, such as a probability, between the pattern related features (e.g., the data structure or set of aforementioned features such as temporal features and/or morphological features as previously described) that are associated with particular seed events and features of known sleep disordering event related patterns, to determine whether the particular seed events are indicative of sleep disordering events or not. In other words, the classifier may decide that one or more sets of seed events may be associated with respiratory related sleep disordering events while another set of seed events may be associated with non-respiratory related sleep disordering events, based on the patterns. The classifier(s), such as its model, may be any one or more of a decision tree classifier or model, a machine learning classifier or model, a logistic regression classifier or model, a neural network, Naive Bayes classifier or model, and a support vector machine, etc. Thus, the determined degree(s) of fit may comprise, for example, activation value(s) or objective measure(s) of fit and may involve output computation(s) of one or more activation functions when applied to the values of the input features and/or may, for example, involve computation of probabilistic value(s) that determine a similarity of the features to the patterns, which probabilistic values may be compared to one or more thresholds for confirming the identification(s) (e.g., of the respiratory or non-respiratory events).

Optionally, multiple classifiers may be implemented. For example, a first classifier may be trained to perform the previously described sequencing related pattern evaluation of the seed events from the plurality of physiological signals that relates the seed events, and a second classifier may be trained then to detect, from the sequences of seed events, the seed events that are associated with respiratory related sleep disordering events, or the seed events that are associated with non-respiratory related sleep disordering events. The distinction between respiratory related sleep disordering events and non-respiratory related sleep disordering events may, for example, be based on specific characteristics of the patterns and of the sequence itself. As an example, a periodic limb movement sequence of seed events, which is a non-respiratory related sleep disordering event, would not have oxygen desaturations and would have a relatively short time between subsequent events. Alternatively, a single classifier may be trained to perform the step of grouping followed by the step of detecting. A classifier may also be trained to perform the detection step without any grouping of seed events into sets. The classifier may for example have been trained by labelled data sets coming from clinical trials. Alternatively, a classifier may have been trained or developed as a rules-based system based on threshold values of any of the above-mentioned features. The classifier can be any known classifier such as a neural network, a decision tree, or a support vector machine.

In this manner, particular seed events may be selected by the processing algorithm based on the determined degrees of fit with the aforementioned patterns such with the aforementioned features, for example, based on a substantially regular duration of seed events in a physiological signal, and taken as a detection of sleep disordering events based on significance of the degree of fit, and, as such, sleep disordering events can be detected with a relatively high degree of confidence, thereby improving previous detection technologies such as ones involving home study devices that may be based on PPG sensing. The classifier may be trained or developed by designing and inputting a set of rules for the detecting. These rules may for example include rules based on threshold values for one or more measurements. These rules can also include rules based on one or more of the determined values linked to the temporal and/or morphological pattern. As an example, the classifier may use as input, among others, information related to the characterization of the seed events, the degrees of fit of the seed events with the patterns, such as substantially regular patterns, and the characterization of the patterns. In some implementations, the classifier may also be trained by using labelled data based on previous measurements. A training dataset may, for example, be constructed from empirical data of a clinical trial in which by means of manual or computer-aided scoring, the location of sleep disordering events has been scored or annotated and to which seed events can be associated. Alternatively, or additionally, a training dataset may for example be constructed from empirical data of a clinical trial in which by means of manual or computer-aided scoring, seed events have been labelled as belonging to a sleep disordering event or not.

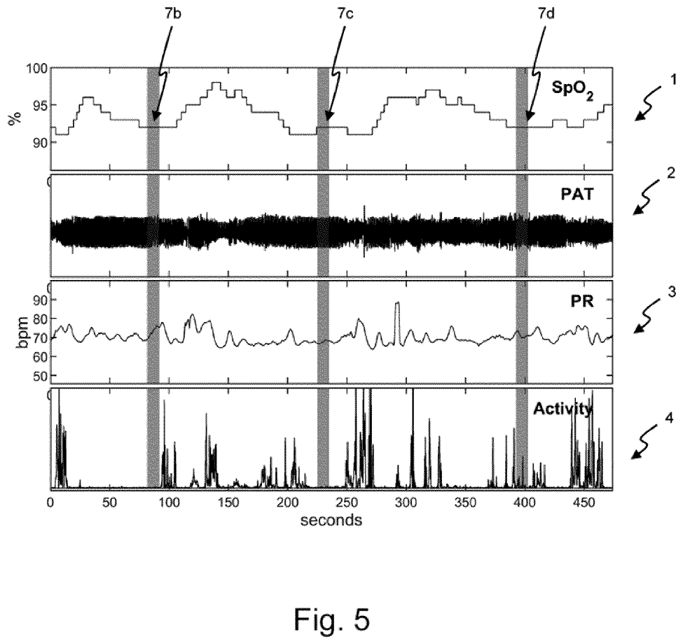

Such improved processes may be considered in relation to FIGS. 4 and 5. FIG. 4 shows a third schematic graph representing of a plurality of physiological signals over time. As in the previous figures, the graphs represent a plurality of physiological signals spanning a common time span, including a saturation of oxygen (SpO₂) signal 1, a PAT signal 2, a pulse rate signal 3 and a limb activity signal 4. In the SpO₂ signal 1, five desaturations of oxygen 7, 7a can be distinguished, which can be selected as being five seed events. In prior art method, an amplitude of these desaturations would be determined and compared to threshold values in order to decide whether or not these events may be associated with sleep disordering events. In the present graph, the first desaturation of oxygen 7a would probably be disqualified given the small difference of only 2% with respect to the previous baseline. However, in an example process according to the present technology, features concerning the substantially regular pattern in for example the onset of the desaturation, and/or in the duration of the seed event, would cause the process to affirmatively detect a sleep disordering event, for example an apnea, in spite of a relatively small oxygen desaturation value. Moreover, the process of evaluating features that serve to group seed events into the aforementioned sets of seed events (e.g., sequences) between different physiological signals strengthen a level of confidence of this detection since there is a substantially regular pattern in a co-occurrence of the seed events of the set, in particular a PAT signal amplitude drop 8a from a baseline, a pulse rate signal amplitude increase 9a from a baseline, and an increase in limb activity 10a, which all temporally align according to said substantially regular pattern.

FIG. 5 shows a fourth schematic graph representing of a plurality of physiological signals 1, 2, 3, 4 over time. Again, in the SpO₂ signal 1, three desaturations of oxygen 7b, 7c, 7d can be distinguished, which can be selected as being three seed events being potentially indicatory of sleep disordering events. In a prior art method, an amplitude of these desaturations would be determined and compared to threshold values. All oxygen desaturation events 7c,7d, and 7b would exceed the threshold value, so the method would decide that a sleep disordering event has been detected. However, in a process of the present technology, features regarding substantially regular patterns would be evaluated within at least part of the seed events. This would prove to be difficult (so as to result in computation of a low or insignificant degree of fit) since even the oxygen desaturation signal 1, it is difficult to recognize a pattern, every desaturation 7b, 7c, 7d having a different overall shape. In the other physiological signals 2, 3 and 4, the situation is still more convincing since there does not seem to be any substantially regular pattern in spite of some PAT amplitude decreases, pulse rate spikes or limb activity spikes. An example process of the present technology would therefore indicate that no sleep disordering event has been detected, and that the desaturations may have been artefacts and/or due to another cause than a sleep disordering event.

Thus, in some implementations of such a process, the apparatus may be configured to generate feedback about a detection of a sleep disordering event or not in response to an input to a system that selects at least one seed event such as when the selection is input into a user interface of such a system. In response, the process may output an indication of a reason why a seed event had not been identified as a sleep disordering event (e.g., a sleep disordered breathing event), for example indicating “absence of substantially regular patterns”. The feedback can preferably be rendered in natural language. Positive feedback may, for example, include “detection based on high degree of fit with a regular pattern in spite of low desaturation”, which might apply to the physiological signals described in FIG. 4 if applied to such a process.

In this regard, the method may include feedback on a detection of a sleep disordering event or not by selection of at least one seed event. The feedback may be rendered in natural language. The feedback may for example include information on why a seed event had been assessed as being associated with a sleep disordering event or had been assessed as not being associated with a sleep disordering event. The information may, for example, be based on a decision tree classifier. The following additional Examples can therefore also be seen as examples of reasoning of the decision tree classifier, which feedback can, for example, be shown as coded sentences on a screen.

Example: A respiratory related sleep disordering event may for example have been detected and the feedback may include information such as “Even though the oxygen desaturation associated with this event is less than 3%, it is part of a train of desaturation events with a relatively large temporal and morphological similarity. The neighbouring two oxygen desaturations, which are part of this train, are on average larger than 3%. The neighbouring six oxygen desaturations, which are a part of this train, are on average larger than 3.5%. The desaturations overlapped largely with trains of pulse rate spikes and PAT reductions with a very similar periodicity.”

Example: A respiratory related sleep disordering event may for example have been detected and the feedback may include information such as “The oxygen desaturation associated with this event is part of a train of oxygen desaturation events with a relatively large temporal and morphological similarity. The neighbouring two oxygen desaturations, which are part of this train, are on average larger than 3%. The neighbouring six oxygen desaturations, which are a part of this train, are on average larger than 3.5%. The limb motion surrounding this event was very low”.

Example: In case a seed event has not been selected or withheld as being associated to a sleep disordering event, the feedback may include information such as “Even though the neighbouring two oxygen desaturations are on average larger than 3% and the neighbouring six oxygen desaturations are on average larger than 3.5%, there was too much limb movement around this location and the surrounding oxygen desaturations had low temporal and morphological similarity”.

The language or wording of such feedback information need of course not be the same as for the present application. However, it will be clear to the person skilled in the art that the ‘temporal and morphological similarity’ refer to the degrees of fit to the patterns in the seed events. Other ways of rendering this information are possible as well.

As has been shown in the previous examples, the present method for detecting sleep disordering events can improve accuracy of the detection of sleep disordering events, either based on a single, or a plurality of, physiological signals in that the process relies on contextual information around a single seed event in the form of determining patterns, such as substantially regular patterns, and determining degrees of fit with said patterns rather than solely relying upon a decision tree based on threshold values for isolated seed events, as is done in prior art methods.

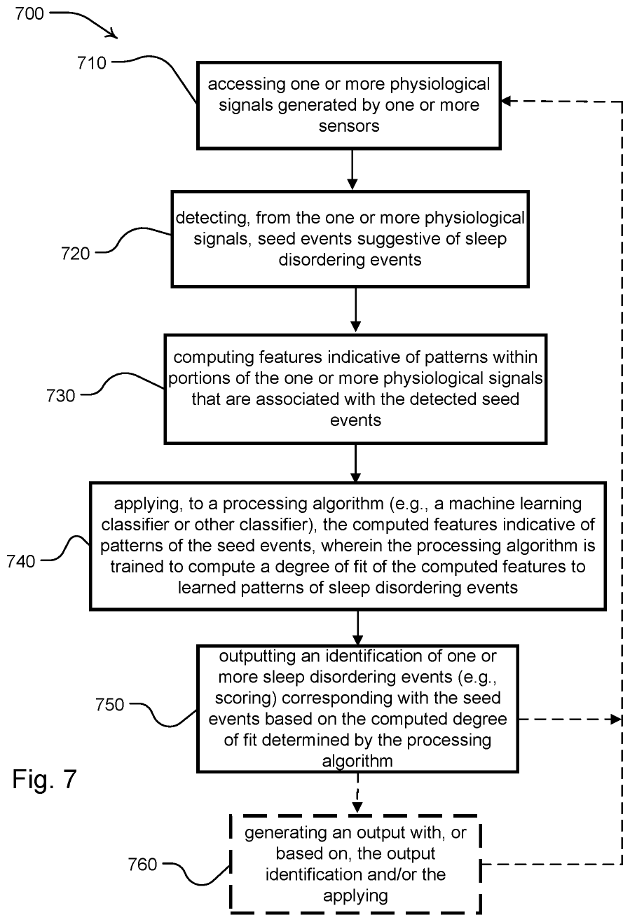

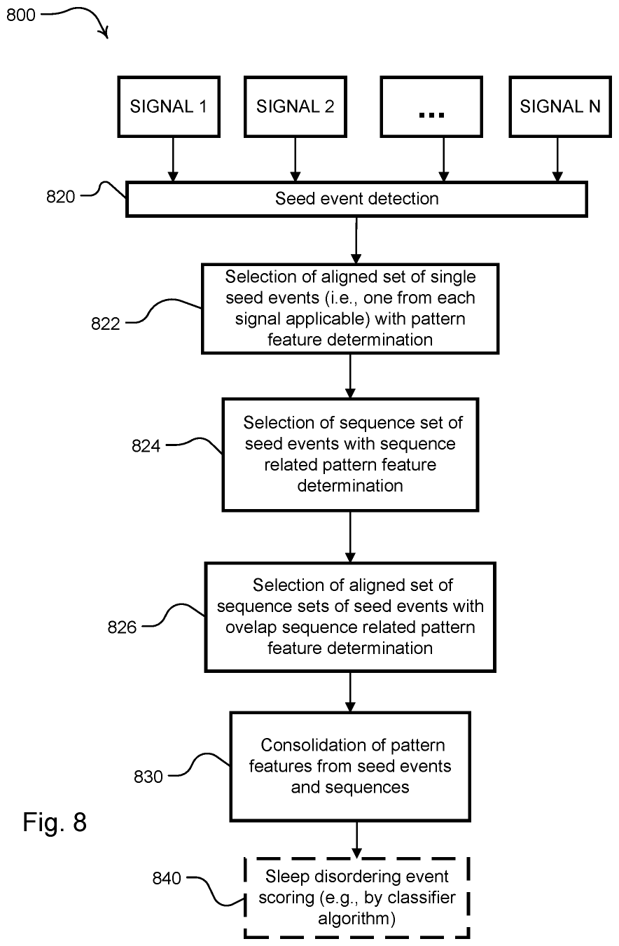

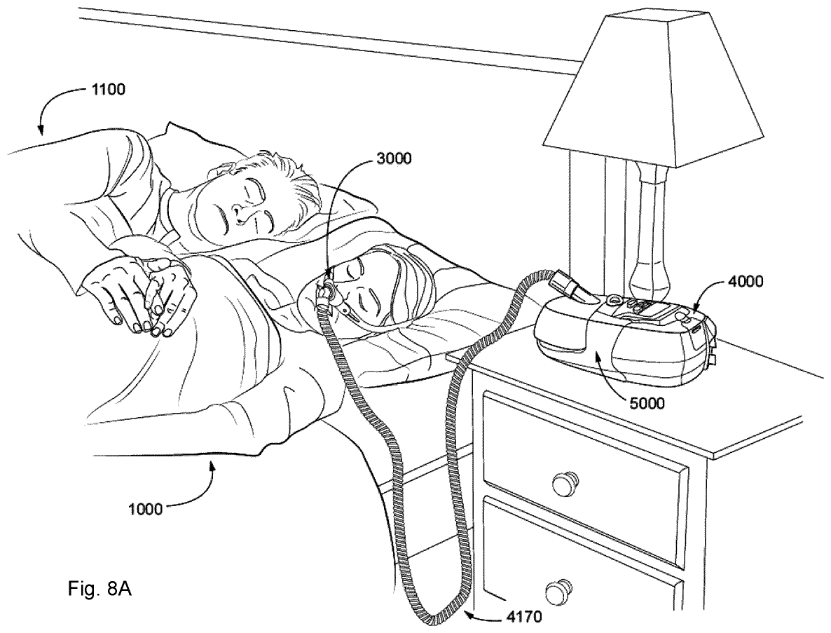

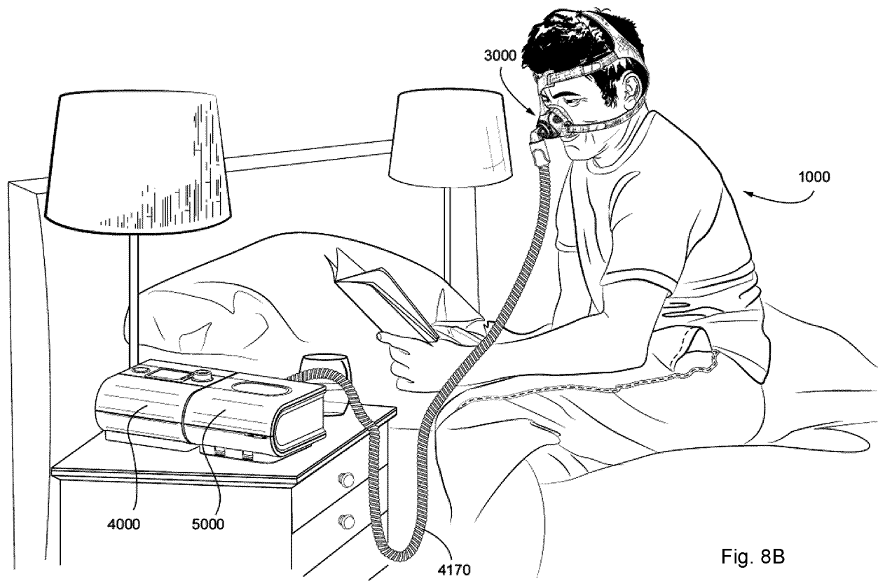

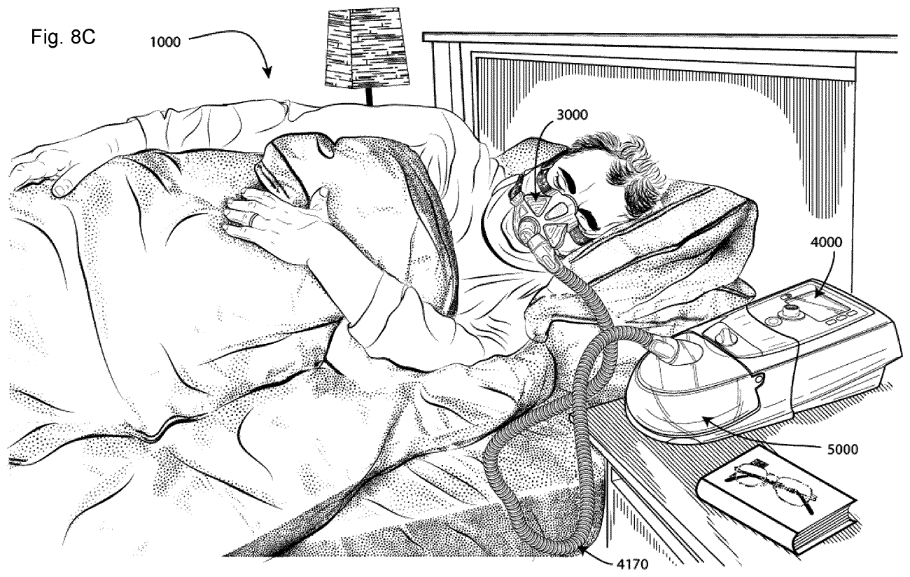

In this regard, such methodologies of the present technology as previously described may be further considered in relation to the system process flow charts of FIGS. 7 and 8. In FIG. 7, the process 700 may begin at 710. In 710, one or more processors may access or receive one or more physiological signals generated by one or more sensors, such as any one or more of the signals and sensors described herein, including for example, a PAT signal. At 720, the one or more processors may detect, from the one or more physiological signals, seed events suggestive of sleep disordering events. At 730, the one or more processors may compute features indicative of patterns, or repetitive patterns, within portions of the one or more physiological signals that are associated with the detected seed events. At 740, the one or more processors may apply, to a processing algorithm (e.g., a machine learning classifier or other classifier or deterministic process such as a hard coded process or decision tree), the computed features indicative of patterns of the seed events. The processing algorithm may be trained to compute a degree of fit of the computed features to learned patterns of sleep disordering events. At 750, the one or more processors may output an identification of one or more sleep disordering events (e.g., scoring) corresponding with the seed events based on the computed degree of fit determined by the processing algorithm. Optionally, at 760, one or more processors of the system may generate an output with, or based on, the output identification and/or the applying. Such an output may include, for example, a signal, such as control signal, with a setting or for setting (e.g., a pressure or flow rate setting) of an operation of a respiratory therapy, such as the operations or therapy operations described further detail herein. Such as signal may optionally be communicated to the respiratory therapy device from the one or more processors, such via a communications network (e.g., an internet) and/or other intermediary device(s) (e.g., one or more servers).

An example of such a process may be further considered in relation to the flow chart of FIG. 8. Similar to FIG. 7, at 820 of process 800, the one or more processors may detect, from one or more physiological signals, seed events suggestive of sleep disordering events. At 822, the one or more processors may select an aligned or related set of signal seed events, such as one from each signal, and may compute or determine one or more features of each event and/or relating such seed events. At 824, the one or more processors may select one or more sequence sets of seed events and may compute or determine one or more sequence related pattern features. At 826, the one or more processors may select one or more aligned or related sets of sequence sets of seed events and may compute or determine overlap sequence related pattern features. At 830, the one or more processors may then consolidate the aforementioned pattern related features from the seed events and sequences. At 840, the one or more processors may then score a sleep disordering event with the consolidated features, such as by applying the features to a classifier algorithm, such any one or more of the classifier algorithms previously described. Further output based on the score may then be generated as previously described.

FIG. 6B shows a suitable computing system 600 comprising circuitry enabling the performance of any one or more of the processes, or any one or more of the step(s) therein, according to the described examples of the present technology such as with the sensor apparatus described herein, such as the finger sensor apparatus of FIG. 6A. Computing system 600 may in general be formed as a suitable general-purpose computer and comprise a bus 610, a processor 602, a local memory 604, one or more optional input interfaces 614, one or more optional output interfaces 616, a communication interface 612, a storage element interface 606, and one or more storage elements 608. Bus 610 may comprise one or more conductors that permit communication among the components of the computing system 600. Processor 602 may include any type of conventional processor or microprocessor that interprets and executes programming instructions. Local memory 604 may include a random-access memory (RAM) or another type of dynamic storage device that stores information and instructions for execution by processor 602 and/or a read only memory (ROM) or another type of static storage device that stores static information and instructions for use by processor 602. Input interface 614 may comprise one or more conventional mechanisms that permit an operator or user to input information to the computing device 600, such as a keyboard 620, a mouse 630, a pen, voice recognition and/or biometric mechanisms, a camera, etc. Output interface 816 may comprise one or more mechanisms that output information to the operator or user, such as a display 640, etc. Communication interface 612 may comprise any transceiver-like mechanism such as for example one or more Ethernet interfaces that enables computing system 600 to communicate with other devices and/or systems, for example with other computing devices 681, 682, 683. The communication interface 612 of computing system 600 may be connected to such another computing system by means of a local area network (LAN) or a wide area network (WAN) such as for example the internet. Storage element interface 606 may comprise a storage interface such as for example a Serial Advanced Technology Attachment (SATA) interface or a Small Computer System Interface (SCSI) for connecting bus 610 to one or more storage elements 608, such as one or more local disks, for example SATA disk drives, and control the reading and writing of data to and/or from these storage elements 608. Although the storage element(s) 608 above is/are described as a local disk, in general any other suitable computer-readable media such as a removable magnetic disk, optical storage media such as a CD or DVD,-ROM disk, solid state drives, flash memory cards, etc. may be implemented.

As used in this application, the term “circuitry” may refer to one or more or all of the following:
(a) hardware-only circuit implementations such as implementations in only analog and/or digital circuitry and
(b) combinations of hardware circuits and software, such as (as applicable):
(i) a combination of analog and/or digital hardware circuit(s) with software/firmware and
(ii) any portions of hardware processor(s) with software (including digital signal processor(s)), software, and memory(ies) that work together to cause an apparatus, such as a mobile phone or server, to perform various functions) and
(c) hardware circuit(s) and/or processor(s), such as microprocessor(s) or a portion of a microprocessor(s), that requires software (e.g. firmware) for operation, but the software may not be present when it is not needed for operation.

This definition of circuitry applies to all uses of this term in this application, including in any claims. As a further example, as used in this application, the term circuitry also covers an implementation of merely a hardware circuit or processor (or multiple processors) or portion of a hardware circuit or processor and its (or their) accompanying software and/or firmware. The term circuitry also covers, for example and if applicable to the particular claim element, a baseband integrated circuit or processor integrated circuit for a mobile device or a similar integrated circuit in a server, a cellular network device, or other computing or network device.

Thus, a processing device that may implement any of the aforementioned processes may include integrated chips, a memory and/or other control instruction, data or information storage medium. For example, programmed instructions encompassing any of the assessment/signal processing methodologies described herein may be coded on integrated chips in the memory of the device or apparatus to form an application specific integrated chip (ASIC). Such instructions, with such processes, may also or alternatively be loaded as software or firmware using an appropriate processor-readable medium(s), data storage medium or memory. Optionally, such processing instructions may be downloaded such as from a server over a network (e.g., an internet) to a processing device such that when the instructions are executed, the processing device serves as a screening, monitoring device and/or treatment device.

Accordingly, such a processing device, such as a server, may include a number of components such as an interface to link with the aforementioned sensors and/or receive data representing such signals from such sensors. The processing device may also include, among other components, a processor(s), an optional display interface, an optional user control/input interface, and a memory/data storage 312, such as with the processing instructions of the processing methodologies/modules described herein. One or more of the sensors may be integral with or operably coupled with processing device. For example, sensor(s) may be integral with processing device or coupled with processing device such as through a wired or wireless link (e.g., Bluetooth, Wi-Fi etc.). Thus, the processing device may include a data communications interface. In some implementations, a system of the present technology may include a server. The server may be configured with access to any of the processor-readable medium(s) or data storage medium(s) described herein that encompasses processor control instructions of any of the aforementioned processes. The server may be configured to receive requests for downloading the processor-executable instructions of the processor-readable medium to such processing devices over a network. In some implementations, the processing device may be a smart phone, tablet or other smart device, or other computing device. The processing device may be a respiratory therapy device, such as a respiratory therapy device described herein that operates to provide a therapy using a controller of a blower.

### 4.1 Optional Example Treatment Systems

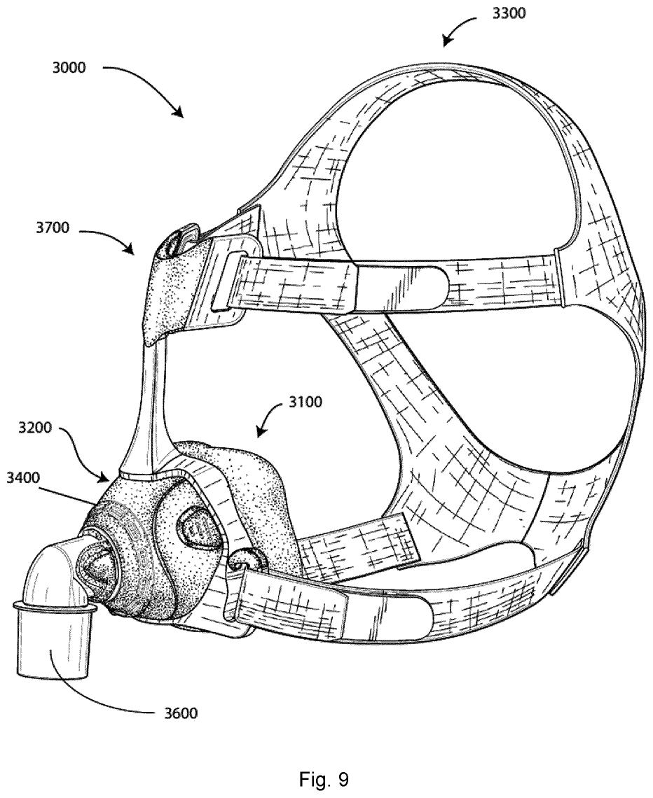

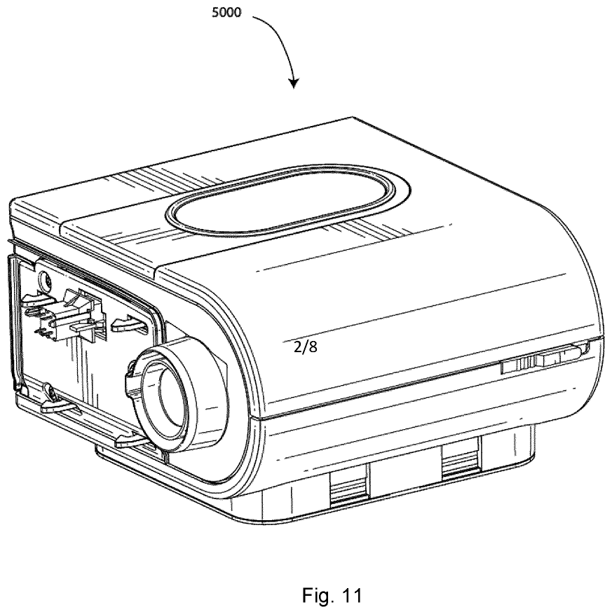

As previously mentioned, in one form, the present technology may include an apparatus or device for treating and/or monitoring a respiratory disorder, which may be involved in and/or configured for any of the aforementioned processes so as to serve as, or be in communication with, the previously described computing system of FIG. 6. The apparatus or device may be a respiratory therapy device (RT) such as an RPT device 4000 for supplying a flow of pressurised air to the patient 1000 via an air circuit 4170 leading to a patient interface 3000. The flow of air may be pressure-controlled (for respiratory pressure therapies) or flow-controlled (for flow therapies such as high flow therapy HFT). Thus, RPT devices may also be configured to act as flow therapy devices, such as when using a patient interface that does not use a seal that seals with the patient's respiratory system. In the following description, the RT or RPT device may be considered in reference to FIGS. 8-11.

### 4.2 Patient Interface

A non-invasive patient interface 3000 in accordance with one aspect of the present technology may optionally comprise any of the following functional aspects: a seal-forming structure 3100, a plenum chamber 3200, a positioning and stabilising structure 3300, a vent 3400, a connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to an airway of the patient so as to facilitate the supply of pressurised air to the airway.

### 4.3 RPT Device

An RPT device 4000 in accordance with one aspect of the present technology comprises mechanical and pneumatic components 4100, electrical components 4200 and is programmed to execute one or more algorithms 4300. The RPT device 4000 may have an external housing 4010 formed in two parts, an upper portion 4012 and a lower portion 4014. In one form, the external housing 4010 may include one or more panel(s) 4015. The RPT device 4000 may comprise a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying pressurised air (e.g., a blower 4142), an outlet muffler 4124, and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 4.3.1 RPT Device Mechanical & Pneumatic Components

An RPT device 4000 may comprise one or more of the following components in an integral unit. In an alternative form, one or more of the following components may be located as respective separate units.

### 4.3.1.1 Air Filter(s)

An RPT device 4000 in accordance with one form of the present technology may include an air filter 4110, or a plurality of air filters 4110.

In one form, an air inlet filter 4112 is located at the beginning of the pneumatic path upstream of a pressure generator 4140.

In one form, an air outlet filter 4114, for example an antibacterial filter, is located between an outlet of the pneumatic block 4020 and a patient interface 3000.

### 4.3.1.2 Muffler(s)

An RPT device 4000 in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000.

### 4.3.1.3 Pressure Generator

In one form of the present technology, a pressure generator 4140 for supplying pressurised air is a controllable blower 4142. For example, the blower 4142 may include a brushless DC motor 4144 with one or more impellers housed in a volute. The pressure generator 4140 may be capable of generating a supply or flow of air, for example at about 120 litres/minute, at a positive pressure in a range from about 4 cmH₂O to about 20 cmH₂O, or in other forms up to about 30 cmH₂O.

The pressure generator 4140 is under the control of the therapy device controller 4240.

In other forms, a pressure generator 4140 may be a piston-driven pump, a pressure regulator connected to a high pressure source (e.g., compressed air reservoir), or a bellows.

### 4.3.1.4 Transducer(s)

Transducers may be internal of the RPT device, or external of the RPT device, such as a finger PPG sensor or PAT sensing device previously described, including, for example, a finger sensor apparatus as illustrated in FIG. 6A. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 are constructed and arranged to generate data representing respective properties of the air flow, such as a flow rate, a pressure or a temperature, at that point in the pneumatic path.

In one form of the present technology, one or more transducers 4270 are located proximate to the patient interface 3000.

In one form, a signal from a transducer 4270 may be filtered, such as by low-pass, high-pass or band-pass filtering.

### 4.3.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

4.3.1.6 Air circuit

An air circuit 4170 in accordance with one aspect of the present technology is a conduit or tube constructed and arranged to allow, in use, a flow of air to travel between two components such as the pneumatic block 4020 and the patient interface 3000.

### 4.3.1.7 Oxygen Delivery

In one form of the present technology, supplemental oxygen 4180 is delivered to one or more points in the pneumatic path, such as upstream of the pneumatic block 4020, to the air circuit 4170 and/or to the patient interface 3000.

### 4.3.2 RPT Device Electrical Components

### 4.3.2.1 Power Supply

In one form of the present technology power supply 4210 is internal of the external housing 4010 of the RPT device 4000. In another form of the present technology, power supply 4210 is external of the external housing 4010 of the RPT device 4000.

In one form of the present technology power supply 4210 provides electrical power to the RPT device 4000 only. In another form of the present technology, power supply 4210 provides electrical power to both RPT device 4000 and humidifier 5000.

### 4.3.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form the input device 4220 may be constructed and arranged to allow a person to select a value and/or a menu option.

### 4.3.2.3 Central Controller

In one form of the present technology, the central controller 4230 is a processor suitable to control an RPT device 4000 such as an x86 INTEL processor.

A central controller 4230 suitable to control an RPT device 4000 in accordance with another form of the present technology includes a processor based on ARM Cortex-M processor from ARM Holdings. For example, an STM32 series microcontroller from ST MICROELECTRONICS may be used.

Another central controller 4230 suitable to control an RPT device 4000 in accordance with a further alternative form of the present technology includes a member selected from the family ARM9-based 32-bit RISC CPUs. For example, an STR9 series microcontroller from ST MICROELECTRONICS may be used.

In certain alternative forms of the present technology, a 16-bit RISC CPU may be used as the central controller 4230 for the RPT device 4000. For example a processor from the MSP430 family of microcontrollers, manufactured by TEXAS INSTRUMENTS, may be used.

In another form of the present technology, the central controller 4230 is a dedicated electronic circuit. In another form, the central controller 4230 is an application-specific integrated circuit (ASIC). In another form, the central controller 4230 comprises discrete electronic components.

The central controller 4230 is configured to receive input signal(s) from one or more transducers 4270, one or more input devices 4220, and the humidifier 5000.

The central controller 4230 is configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms 4300, expressed as computer programs stored in a non-transitory computer readable storage medium, such as memory 4260 or other memory described herein. In some forms of the present technology, as previously discussed, the central controller 4230 may be integrated with an RPT device 4000. However, in some forms of the present technology, some methodologies may be performed by a remotely located device or server such as the server previously mentioned. For example, the remotely located device or server may determine control settings for transfer to a ventilator or other RT device such as by detecting respiratory related events and distinguishing them by type by an analysis of stored data such as from any of the sensors described herein.

While the central controller 4230 may comprise a single controller interacting with various sensors 4270, data communications interface 4280, memory 4260, as well as other devices, the functions of controller 4230 may be distributed among more than one controller. Thus, the term “central” as used herein is not meant to limit the architecture to a single controller or processor that controls the other devices. For example, alternative architectures may include a distributed controller architecture involving more than one controller or processor, which may optionally be directly or indirectly in electronic (wired or wireless) communications with the previously described finger sensor or a server in communication with the finger sensor, such as for implementing any of the methodologies described herein. This may include, for example, a separate local (i.e., within RPT device 4000) or remotely located controller that perform some of the algorithms 4300, or even more than one local or remote memory that stores some of the algorithms. In addition, the algorithms when expressed as computer programs may comprise high level human readable code (e.g., C++, Visual Basic, other object oriented languages, etc.) or low/machine level instructions (Assembler, Verilog, etc.). Depending on the functionality of an algorithm(s), such code or instructions may be burnt in the controller, e.g., an ASIC or DSP, or be a run time executable ported to a DSP or general purpose processor that then becomes specifically programmed to perform the tasks required by the algorithm(s).

### 4.3.2.4 Clock

The RPT device 4000 may include a clock 4232 that is connected to the central controller 4230.

### 4.3.2.5 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 4.3.2.6 Protection Circuits

An RPT device 4000 in accordance with the present technology may comprise one or more protection circuits 4250.

One form of protection circuit 4250 in accordance with the present technology is an electrical protection circuit.

One form of protection circuit 4250 in accordance with the present technology is a temperature or pressure safety circuit.

### 4.3.2.7 Memory

In accordance with one form of the present technology the RPT device 4000 includes memory 4260, for example non-volatile memory. In some forms, memory 4260 may include battery powered static RAM. In some forms, memory 4260 may include volatile RAM.

Memory 4260 may be located on PCBA 4202. Memory 4260 may be in the form of EEPROM, or NAND flash.

Additionally or alternatively, RPT device 4000 includes a removable form of memory 4260, for example a memory card made in accordance with the Secure Digital (SD) standard.

In one form of the present technology, the memory 4260, such as an of the memories previously described, acts as a non-transitory computer readable storage medium on which is stored computer program instructions expressing the one or more methodologies described herein, such as the one or more algorithms 4300.

### 4.3.2.8 Transducers

Transducers may be internal of the device 4000, or external of the RPT device 4000. External transducers may be located for example on or form part of the air delivery circuit 4170, e.g., at the patient interface 3000. External transducers may be in the form of non-contact sensors such as a Doppler radar movement sensor that transmit or transfer data to the RPT device 4000.

### 4.3.2.8.1 Flow Rate

A flow rate transducer 4274 in accordance with the present technology may be based on a differential pressure transducer, for example, an SDP600 Series differential pressure transducer from SENSIRION. The differential pressure transducer is in fluid communication with the pneumatic circuit, with one of each of the pressure transducers connected to respective first and second points in a flow restricting element.

In one example, a signal representing total flow rate Qt from the flow transducer 4274 is received by the central controller 4230.

### 4.3.2.8.2 Pressure

A pressure transducer 4272 in accordance with the present technology is located in fluid communication with the pneumatic path. An example of a suitable pressure transducer 4272 is a sensor from the HONEYWELL ASDX series. An alternative suitable pressure transducer is a sensor from the NPA Series from GENERAL ELECTRIC.

In use, a signal from the pressure transducer 4272 is received by the central controller 4230. In one form, the signal from the pressure transducer 4272 is filtered prior to being received by the central controller 4230.

### 4.3.2.8.3 Motor Speed

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 4.3.2.9 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g., via Ethernet, or optical fibre) or a wireless protocol (e.g., CDMA, GSM, LTE) to connect to the Internet.

In one form, local external communication network 4284 utilises one or more communication standards, such as Bluetooth, or a consumer infrared protocol and may optionally communicate with any of the sensors described herein.

In one form, remote external device 4286 is one or more computers, for example a cluster of networked computers and/or server as described herein. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 4.3.2.10 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 4.3.2.10.1 Display Driver

A display driver 4292 receives as an input the characters, symbols, or images intended for display on the display 4294, and converts them to commands that cause the display 4294 to display those characters, symbols, or images.

### 4.3.2.10.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 4.3.3 RPT Device Algorithms

### 4.3.3.1 Pre-Processing Module

A pre-processing module 4310 in accordance with the present technology receives, as an input, raw data from a transducer 4270, for example a flow rate sensor 4274 or a pressure sensor 4272, and performs one or more process steps to calculate one or more output values that will be used as an input to another module, for example a therapy engine module 4320.

In one form of the present technology, the output values include the interface or mask pressure Pm, the respiratory flow rate Qr, and the leak flow rate Ql.

In various forms of the present technology, the pre-processing module 4310 comprises one or more of the following algorithms: pressure compensation 4312, vent flow rate estimation 4314, leak flow rate estimation 4316, respiratory flow rate estimation 4317, ventilation determination 4311, target ventilation determination 4313, respiratory rate estimation 4318, and backup rate determination 4319.

### 4.3.3.1.1 Pressure Compensation

In one form of the present technology, a pressure compensation algorithm 4312 receives as an input a signal indicative of the pressure in the pneumatic path proximal to an outlet of the pneumatic block 4020. The pressure compensation algorithm 4312 estimates the pressure drop in the air circuit 4170 and provides as an output an estimated pressure, Pm, in the patient interface 3000.

### 4.3.3.1.2 Vent Flow Rate Estimation

In one form of the present technology, a vent flow rate estimation algorithm 4314 receives as an input an estimated pressure, Pm, in the patient interface 3000 and estimates a vent flow rate of air, Qv, from a vent 3400 in a patient interface 3000.

### 4.3.3.1.3 Leak Flow Rate Estimation

In one form of the present technology, a leak flow rate estimation algorithm 4316 receives as an input a total flow rate Qt and a vent flow rate Qv, and estimates a leak flow rate Ql. In one form, the leak flow rate estimation algorithm 4316 estimates the leak flow rate Ql by calculating an average of the difference between the total flow rate and the vent flow rate Qv over a period sufficiently long to include several breathing cycles, e.g., about 10 seconds.

In one form, the leak flow estimation algorithm 4316 receives as an input a total flow rate Qt, a vent flow rate Qv, and an estimated pressure, Pm, in the patient interface 3000, and estimates a leak flow rate Ql by calculating a leak conductance, and determining a leak flow rate Ql to be a function of leak conductance and the pressure Pm. Leak conductance may be calculated as the quotient of low-pass filtered non-vent flow rate equal to the difference between total flow rate Qt and vent flow rate Qv, and low-pass filtered square root of pressure Pm, where the low-pass filter time constant has a value sufficiently long to include several breathing cycles, e.g., about 10 seconds. The leak flow rate Ql may be estimated as the product of leak conductance and a function of pressure, Pm.

### 4.3.3.1.4 Respiratory Flow Rate Estimation

In one form of the present technology, a respiratory flow rate estimation algorithm 4317 receives as an input a total flow rate, Qt, a vent flow rate, Qv, and a leak flow rate, Ql, and estimates a respiratory flow rate of air, Qr, to the patient, by subtracting the vent flow rate Qv and the leak flow rate Ql from the total flow rate Qt.

In other forms of the present technology, the respiratory flow estimation algorithm 4317 provides a value that acts as a proxy for the respiratory flow rate Qr. Possible proxies for respiratory flow rate include:
Respiratory movement of the chest of the patient 1000
Current drawn by the pressure generator 4140
Motor speed of the pressure generator 4140
Trans-thoracic impedance of the patient 1000

The respiratory flow rate proxy value may be provided by a transducer 4270 in the RPT device 4000, e.g., the motor speed sensor 4276, or a sensor external to the RPT device 4000, such a respiratory movement sensor or a trans-thoracic impedance sensor.

### 4.3.3.1.5 Ventilation Determination

In one form of the present technology, a ventilation determination algorithm 4311 receives an input a respiratory flow rate Qr, and determines a measure Vent indicative of current patient ventilation.

In some implementations, the ventilation determination algorithm 4311 determines a measure of ventilation Vent that is an estimate of actual patient ventilation.

In one such implementation, the measure of ventilation Vent is half the absolute value of respiratory flow, Qr, optionally filtered by low-pass filter such as a second order Bessel low-pass filter with a corner frequency of 0.11 Hz.

In one such implementation, the measure of ventilation Vent is an estimate of gross alveolar ventilation (i.e. non-anatomical-deadspace ventilation). This requires an estimate of anatomical deadspace. One can use the patient's height (or arm-span in cases of severe skeletal deformity) as a good predictor of anatomical deadspace. Gross alveolar ventilation is then equal to a measure of actual patient ventilation, e.g., determined as above, less the product of the estimated anatomical deadspace and the estimated spontaneous respiratory rate Rs.

In other implementations, the ventilation determination algorithm 4311 determines a measure of ventilation Vent that is broadly proportional to actual patient ventilation. One such implementation estimates peak respiratory flow rate Qpeak over the inspiratory portion of the cycle. This and many other procedures involving sampling the respiratory flow rate Qr produce measures which are broadly proportional to ventilation, provided the flow rate waveform shape does not vary very much (here, the shape of two breaths is taken to be similar when the flow rate waveforms of the breaths normalised in time and amplitude are similar). Some simple examples include the median positive respiratory flow rate, the median of the absolute value of respiratory flow rate, and the standard deviation of flow rate. Arbitrary linear combinations of arbitrary order statistics of the absolute value of respiratory flow rate using positive coefficients, and even some using both positive and negative coefficients, are approximately proportional to ventilation. Another example is the mean of the respiratory flow rate in the middle K proportion (by time) of the inspiratory portion, where 0<K<1. There is an arbitrarily large number of measures that are exactly proportional to ventilation if the flow rate waveform shape is constant.

In other forms, the ventilation determination algorithm 4311 determines a measure Vent of ventilation that is not based on respiratory flow rate Qr, but is a proxy for the current patient ventilation, such as oxygen saturation (SaO₂), or partial pressure of carbon dioxide (PCO₂), obtained from suitable sensors attached to the patient 1000.

### 4.3.3.1.6 Target Ventilation Determination

In one form of the present technology, a central controller 4230 takes as input the measure of current ventilation, Vent, and executes one or more target ventilation determination algorithms 4313 for the determination of a target value Vtgt for the measure of ventilation.

In some forms of the present technology, there is no target ventilation determination algorithm 4313, and the target ventilation Vtgt is predetermined, for example by hard-coding during configuration of the RPT device 4000 or by manual entry through the input device 4220.

In other forms of the present technology, such as adaptive servo-ventilation (ASV) therapy (described below), the target ventilation determination algorithm 4313 computes the target ventilation Vtgt from a value Vtyp indicative of the typical recent ventilation of the patient 1000.

In some forms of adaptive servo-ventilation therapy, the target ventilation Vtgt is computed as a high proportion of, but less than, the typical recent ventilation Vtyp. The high proportion in such forms may be in the range (80%, 100%), or (85%, 95%), or (87%, 92%).

In other forms of adaptive servo-ventilation therapy, the target ventilation Vtgt is computed as a slightly greater than unity multiple of the typical recent ventilation Vtyp.

The typical recent ventilation Vtyp is the value around which the distribution of the measure of current ventilation Vent over multiple time instants over some predetermined timescale tends to cluster, that is, a measure of the central tendency of the measure of current ventilation over recent history. In one implementation of the target ventilation determination algorithm 4313, the recent history is of the order of several minutes, but in any case should be longer than the timescale of Cheyne-Stokes waxing and waning cycles. The target ventilation determination algorithm 4313 may use any of the variety of well-known measures of central tendency to determine the typical recent ventilation Vtyp from the measure of current ventilation, Vent. One such measure is the output of a low-pass filter on the measure of current ventilation Vent, with time constant equal to one hundred seconds.

### 4.3.3.1.7 Respiratory Rate Estimation

In one form of the present technology, a respiratory rate estimation algorithm 4318 receives as an input a respiratory flow rate, Qr, to the patient 1000, and produces an estimate of the spontaneous respiratory rate Rs of the patient.

The respiratory rate estimation algorithm 4318 may estimate the spontaneous respiratory rate Rs over periods when the patient 1000 is breathing spontaneously, i.e., when the RPT device 4000 is not delivering “backup breaths” (described below). In some forms of the present technology, the respiratory rate estimation algorithm 4318 estimates the respiratory rate over periods when servo-assistance (defined as pressure support minus minimum pressure support) is low, in one implementation less than 4 cmH₂O, as such periods are more likely to reflect spontaneous respiratory effort.

In some forms of the present technology, the respiratory rate estimation algorithm 4318 estimates the respiratory rate over periods of asleep breathing, since the respiratory rate during these periods may be substantially different from the respiratory rate during wake. Anxiety typically results in a higher respiratory rate than that prevailing during sleep. When patients focus on their own breathing process, their respiratory rates are typically lower than those during normal wakefulness or during sleep. Techniques such as described in Patent Application no. PCT/AU2010/000894, published as WO 2011/006199, the entire disclosure of which is hereby incorporated herein by reference, may be used to identify periods of awake breathing from the respiratory flow rate, Qr.

In some forms of the present technology, the respiratory rate estimation algorithm 4318 estimates the spontaneous respiratory rate Rs as the reciprocal of one of a variety of well-known statistical measures of central tendency of breath duration Ttot during the period of interest. In such measures it is desirable to reject, or at least be robust to, outliers. One such measure, trimmed mean, in which the lower and upper K proportions of the sorted breath durations are discarded and the mean calculated on the remaining breath durations, is robust to outliers. For example, when K is 0.25, this amounts to discarding the upper and lower quartiles of breath duration Ttot. The median is another robust measure of central tendency, though this can occasionally give unsatisfactory results when the distribution is strongly bimodal. A simple mean may also be employed as a measure of central tendency, though it is sensitive to outliers. An initial interval filtering stage, in which contiguous time intervals corresponding to implausible respiratory rates (e.g., greater than 45 breaths/minute or less than 6 breaths/minute) are excluded as outliers from the mean calculation, may be employed. Other filtering mechanisms which may be used alone or in combination with interval filtering are to exclude any breaths that are not part of a sequence of N successive spontaneous breaths, where N is some small integer (e.g., 3), and to exclude the early and late breaths of a sequence of successive spontaneous breaths, e.g., to exclude the first and last breaths of a sequence of four breaths. The rationale for the latter mechanism is that the first and the last breaths in particular, and the early and late breaths in general, of a sequence of spontaneous breaths may be atypical; for example, the first spontaneous breath may occur as a result of an arousal, and the last spontaneous breath may be longer because of the decreasing respiratory drive which results in the backup breath which ends the sequence of spontaneous breaths.

In some forms of the present technology, the respiratory rate estimation algorithm 4318 makes an initial estimate of the spontaneous respiratory rate Rs using an initial period of estimation, to enable the subsequent processing in the therapy engine module 4320 to begin, and then continuously updates the estimate of the spontaneous respiratory rate Rs using a period of estimation that is longer than the initial period of estimation, to improve statistical robustness. For example, the initial period of estimation may be 20 minutes of suitable spontaneous breaths, but the period of estimation may then progressively increase up to some maximum duration, for example 8 hours. Rather than a rolling window of this duration being used for this estimation, low-pass filters on breath duration may be used, with progressively longer response times (more precisely, progressively lower corner frequencies) as the session proceeds.

In some forms, a suitably processed short-term (e.g., 10-minute) measure of central tendency, such as trimmed mean, may be input to a suitable low-pass filter to give an estimate Rs which changes on the time scale of hours or longer. This has the advantage that potentially large amounts of breath duration data do not need to be stored and processed, as might occur if a trimmed mean needs to be calculated on a moving window of breath duration data lasting hours or days.

In some forms of the present technology, respiratory rates measured over short periods of time, and in particular over one breath, may also be used instead of breath duration in the above-described measures of central tendency, giving generally similar but not identical results.

### 4.3.3.1.8 Backup Rate Determination

In one form of the present technology, a backup rate determination algorithm 4319 receives as input a spontaneous respiratory rate estimate Rs provided by the respiratory rate estimation algorithm 4318 and returns a “backup rate” Rb. The backup rate Rb is the rate at which the RPT device 4000 will deliver backup breaths, i.e., continue to provide ventilatory support, to a patient 1000 in the absence of significant spontaneous respiratory effort.

In one form of the pre-processing module 4310, there is no backup rate determination algorithm 4319, and the backup rate Rb is instead provided manually to the RPT device 4000, e.g., via the input device 4220, or hard-coded at the time of configuration of the RPT device 4000.

In one form, known as adaptive backup rate, the backup rate determination algorithm 4319 determines the backup rate Rb as a function of the spontaneous respiratory rate Rs. In one implementation, the function determines the backup rate Rb as the spontaneous respiratory rate Rs minus a constant such as 2 breaths per minute. In another implementation, the function determines the backup rate Rb as the spontaneous respiratory rate Rs multiplied by a constant that is slightly less than unity.

In one form, known as variable backup rate, the backup rate determination algorithm 4319 determines the backup rate Rb as a function of time. The backup rate Rb is initialised to a value known as the spontaneous backup rate (SBR) that is some fraction of a final target backup rate, known as the sustained timed backup rate (STBR). The fraction may be two thirds, or three quarters, or other positive values less than one. The SBR is the reciprocal of the timeout period to a backup breath when the most recent inspiration was a spontaneous (i.e., patent-triggered) breath. The STBR may be predetermined (e.g., by manual entry or hard-coding as described above) or set to some typical respiratory rate such as 15 bpm. Over time elapsed since the previous spontaneous breath, the backup rate Rb is increased from the SBR towards the STBR. The increase may be according to a predetermined profile, such as a series of steps, or a continuous linear profile. The profile is chosen such that the backup rate Rb reaches the STBR after a predetermined interval. The interval may be measured in units of time, such as 30 seconds, or relative to the patient's respiration, such as 5 breaths.

In some forms of variable backup rate, the predetermined interval over which the backup rate Rb increases from the SBR towards the STBR may be a function of the adequacy of current ventilation. In one implementation, suitable for servo-ventilation in which a target value Vtgt exists for the measure of ventilation, the backup rate approaches the STBR faster to the extent that current measure of ventilation Vent is less than the target ventilation Vtgt.

In one form of variable backup rate, known as adaptive variable backup rate, the backup rate determination algorithm 4319 determines the backup rate Rb as a function of the current estimated spontaneous respiratory rate Rs provided by the respiratory rate estimation algorithm 4318, as well as a function of time. As in variable backup rate determination, adaptive variable backup rate determination increases the backup rate Rb from the SBR towards the STBR over a predetermined interval that may be a function of the adequacy of current ventilation. The STBR may be initialised to a standard respiratory rate, such as 15 bpm. Once a reliable estimate of spontaneous respiratory rate Rs is available from the respiratory rate estimation algorithm 4318, the STBR may be set to the current estimated spontaneous respiratory rate Rs multiplied by some constant. The SBR may be set to some fraction of the STBR, as in variable backup rate. In one form, the fraction, for example two thirds, can be set to a lower value, such as 0.55, during the initial period of estimation of the spontaneous respiratory rate Rs, to accommodate occasional long breath durations in patients with relatively low respiratory rates, such as 12 breaths per minute.

In some forms, the constant by which the current estimated spontaneous respiratory rate Rs is multiplied to obtain the STBR may be slightly higher than 1, e.g., 1.1, to provide more aggressive ventilation during apneas, which may be desirable in short apneas. The constant may be somewhat lower than 1, e.g., 0.8, particularly if difficulty in resynchronisation with the patient on the return of patient effort turns out to be a problem in a particular patient. Lower backup rates make resynchronisation easier, by lengthening the expiratory pause, during which resynchronisation commonly occurs.

### 4.3.3.2 Therapy Engine Module

In one form of the present technology, a therapy engine module 4320 receives as inputs one or more of a pressure, Pm, in a patient interface 3000, a respiratory flow rate of air to a patient, Qr, and an estimate Rs of the spontaneous respiratory rate, and provides as an output one or more therapy parameters. In various forms, the therapy engine module 4320 comprises one or more of the following algorithms: phase determination 4321, waveform determination 4322, inspiratory flow limitation determination 4324, apnea/hypopnea determination 4325, snore detection 4326, airway patency determination 4327, and therapy parameter determination 4329, such as one including a central vs. obstructive type determination as previously described.

### 4.3.3.2.1 Phase Determination

In one form of the present technology, a phase determination algorithm 4321 receives as an input a signal indicative of respiratory flow, Qr, and provides as an output a phase Φ of a current breathing cycle of a patient 1000.

In some forms, known as discrete phase determination, the phase output (D is a discrete variable. One implementation of discrete phase determination provides a bi-valued phase output (D with values of either inhalation or exhalation, for example represented as values of 0 and 0.5 revolutions respectively, upon detecting the start of spontaneous inhalation and exhalation respectively. RPT devices 4000 that “trigger” and “cycle” effectively perform discrete phase determination, since the trigger and cycle points are the instants at which the phase changes from exhalation to inhalation and from inhalation to exhalation, respectively. In one implementation of bi-valued phase determination, the phase output Φ is determined to have a discrete value of 0 (thereby “triggering” the RPT device 4000) when the respiratory flow rate Qr has a value that exceeds a positive threshold, and a discrete value of 0.5 revolutions (thereby “cycling” the RPT device 4000) when a respiratory flow rate Qr has a value that is more negative than a negative threshold.

Another implementation of discrete phase determination provides a tri-valued phase output Φ with a value of one of inhalation, mid-inspiratory pause, and exhalation.

In other forms, known as continuous phase determination, the phase output Φ is a continuous value, for example varying from 0 to 1 revolutions, or 0 to 2π radians. RPT devices 4000 that perform continuous phase determination may trigger and cycle when the continuous phase reaches 0 and 0.5 revolutions, respectively. In one implementation of continuous phase determination, a continuous value of phase Φ is determined using a fuzzy logic analysis of the respiratory flow rate Qr. A continuous value of phase determined in this implementation is often referred to as “fuzzy phase”. In one implementation of a fuzzy phase determination algorithm 4321, the following rules are applied to the respiratory flow rate Qr:
1. If the respiratory flow rate is zero and increasing fast then the phase is 0 revolutions.
2. If the respiratory flow rate is large positive and steady then the phase is 0.25 revolutions.
3. If the respiratory flow rate is zero and falling fast, then the phase is 0.5 revolutions.
4. If the respiratory flow rate is large negative and steady then the phase is 0.75 revolutions.
5. If the respiratory flow rate is zero and steady and the 5-second low-pass filtered absolute value of the respiratory flow rate is large then the phase is 0.9 revolutions.
6. If the respiratory flow rate is positive and the phase is expiratory, then the phase is 0 revolutions.
7. If the respiratory flow rate is negative and the phase is inspiratory, then the phase is 0.5 revolutions.
8. If the 5-second low-pass filtered absolute value of the respiratory flow rate is large, the phase is increasing at a steady rate equal to the patient's respiratory rate, low-pass filtered with a time constant of 20 seconds.

The output of each rule may be represented as a vector whose phase is the result of the rule and whose magnitude is the fuzzy extent to which the rule is true. The fuzzy extent to which the respiratory flow rate is “large”, “steady”, etc. is determined with suitable membership functions. The results of the rules, represented as vectors, are then combined by some function such as taking the centroid. In such a combination, the rules may be equally weighted, or differently weighted.

In another implementation of continuous phase determination, the inhalation time Ti and the exhalation time Te are first estimated from the respiratory flow rate Qr. The phase Φ is then determined as the half the proportion of the inhalation time Ti that has elapsed since the previous trigger instant, or 0.5 revolutions plus half the proportion of the exhalation time Te that has elapsed since the previous cycle instant (whichever was more recent).

In some forms of the present technology, suitable for pressure support ventilation therapy (described below), the phase determination algorithm 4321 is configured to trigger even when the respiratory flow rate Qr is insignificant, such as during an apnea. As a result, the RPT device 4000 delivers “backup breaths” in the absence of spontaneous respiratory effort from the patient 1000. For such forms, known as spontaneous/timed (S/T) modes, the phase determination algorithm 4321 may make use of the backup rate Rb provided by the backup rate determination algorithm 4319.

A phase determination algorithm 4321 that uses “fuzzy phase” may implement S/T mode using the backup rate Rb by including a “momentum” rule in the fuzzy phase rules. The effect of the momentum rule is to carry the continuous phase forward from exhalation to inhalation at the backup rate Rb if there are no features of respiratory flow rate Qr that would otherwise carry the continuous phase forward through the other rules. In one implementation, the more it is true that the measure of ventilation Vent (described below) is well below a target value Vtgt for ventilation (also described below), the more highly the momentum rule is weighted in the combination. However, as a result of the rapid increase in pressure support in response to mild to moderate hypoventilation (with respect to the target ventilation), the ventilation may be quite close to the target ventilation. It is desirable that the momentum rule is given a low weighting when the ventilation is close to target, to allow the patient to breathe at rates significantly lower than the respiratory rate at other times (when the patient is not in a central apnea) without being unnecessarily pushed to breathe at a higher rate by the ventilator. However, when the momentum rule is given a low weighting when ventilation is above a value which is below but close to the target ventilation, adequate ventilation may easily be achieved at a relatively high pressure support at a rate well below the backup rate. It would be desirable for the backup breaths to be delivered at a higher rate, because this would enable the target ventilation to be delivered at a lower pressure support. This is desirable for a number of reasons, a key one of which is to diminish mask leak.

To summarise, in a fuzzy phase determination algorithm 4321 that implements S/T mode, there is a dilemma in choosing the weighting for the momentum rule incorporating the backup rate Rb: if it is too high, the patient may feel “pushed along” by the backup rate. If it is too low, the pressure support may be excessive. Hence it is desirable to provide methods of implementing S/T mode which do not rely on the momentum rule described above.

A phase determination algorithm 4321 (either discrete, or continuous without a momentum rule) may implement S/T mode using the backup rate Rb in a manner known as timed backup. Timed backup may be implemented as follows: the phase determination algorithm 4321 attempts to detect the start of inhalation due to spontaneous respiratory effort, for example by monitoring the respiratory flow rate Qr as described above. If the start of inhalation due to spontaneous respiratory effort is not detected within a period of time after the last trigger instant whose duration is equal to the reciprocal of the backup rate Rb (an interval known as the backup timing threshold), the phase determination algorithm 4321 sets the phase output Φ to a value of inhalation (thereby triggering the RPT device 4000). Once the RPT device 4000 is triggered, and a backup breath begins to be delivered, the phase determination algorithm 4321 attempts to detect the start of spontaneous exhalation, for example by monitoring the respiratory flow rate Qr, upon which the phase output Φ is set to a value of exhalation (thereby cycling the RPT device 4000).

If the backup rate Rb is increased over time from the SBR to the STBR, as in a variable backup rate system described above, the backup timing threshold starts out longer and gradually becomes shorter. That is, the RPT device 4000 starts out less vigilant and gradually becomes more vigilant to lack of spontaneous respiratory effort as more backup breaths are delivered. Such an RPT device 4000 is less likely to make a patient feel “pushed along” if they would prefer to breathe at a lower than standard rate, while still delivering backup breaths when they are needed.

If the STBR in a variable backup rate system adapts to the patient's estimated spontaneous respiratory rate Rs, as in an adaptive variable backup rate system described above, the backup breaths will be delivered at a rate that adapts to the patient's own recent spontaneous respiratory efforts.

### 4.3.3.2.2 Waveform Determination

In one form of the present technology, the therapy control module 4330 controls a pressure generator 4140 to provide a treatment pressure Pt that varies as a function of phase Φ of a breathing cycle of a patient according to a waveform template π(Φ).

In one form of the present technology, a waveform determination algorithm 4322 provides a waveform template π(Φ) with values in the range [0, 1] on the domain of phase values Φ provided by the phase determination algorithm 4321 to be used by the therapy parameter determination algorithm 4329.

In one form, suitable for either discrete or continuously-valued phase, the waveform template π(Φ) is a square-wave template, having a value of 1 for values of phase up to and including 0.5 revolutions, and a value of 0 for values of phase above 0.5 revolutions. In one form, suitable for continuously-valued phase, the waveform template π(Φ) comprises two smoothly curved portions, namely a smoothly curved (e.g., raised cosine) rise from 0 to 1 for values of phase up to 0.5 revolutions, and a smoothly curved (e.g., exponential) decay from 1 to 0 for values of phase above 0.5 revolutions. One example of such a “smooth and comfortable” waveform template is the “shark fin” waveform template, in which the rise is a raised cosine, and the smooth decay is quasi-exponential (so that the limit of π as Φ approaches one revolution is precisely zero).

In some forms of the present technology, the waveform determination algorithm 4322 selects a waveform template π(Φ) from a library of waveform templates, dependent on a setting of the RPT device 4000. Each waveform template π(Φ) in the library may be provided as a lookup table of values H against phase values Φ. In other forms, the waveform determination algorithm 4322 computes a waveform template π(Φ) “on the fly” using a predetermined functional form, possibly parametrised by one or more parameters (e.g., time constant of an exponentially curved portion). The parameters of the functional form may be predetermined or dependent on a current state of the patient 1000.

In some forms of the present technology, suitable for discrete bi-valued phase of either inhalation (Φ=0 revolutions) or exhalation (Φ=0.5 revolutions), the waveform determination algorithm 4322 computes a waveform template π “on the fly” as a function of both discrete phase Φ and time t measured since the most recent trigger instant (transition from exhalation to inhalation). In one such form, the waveform determination algorithm 4322 computes the waveform template π(Φ, t) in two portions (inspiratory and expiratory) as follows:

$$Π ( Φ , t ) = \{ \begin{matrix}{Π}_{i} ( t ) , & Φ = 0 \\ {Π}_{e} ( t - {T}_{i} ) , & Φ = 0.5\end{matrix}$$

where Φᵢ(t) and πₑ(t) are inspiratory and expiratory portions of the waveform template π(Φ, t), and Ti is the inhalation time. In one such form, the inspiratory portion Φᵢ(t) of the waveform template is a smooth rise from 0 to 1 parametrised by a rise time, and the expiratory portion πₑ(t) of the waveform template is a smooth fall from 1 to 0 parametrised by a fall time.

### 4.3.3.2.3 Determination of Inspiratory Flow Limitation

In one form of the present technology, a processor executes one or more algorithms 4324 for the detection of inspiratory flow limitation (partial obstruction).

In one form the algorithm 4324 receives as an input a respiratory flow rate signal Qr and provides as an output a metric of the extent to which the inspiratory portion of the breath exhibits inspiratory flow limitation.

In one form of the present technology, the inspiratory portion of each breath is identified based on the phase Φ estimated at each instant. For example, the inspiratory portion of the breath is the values of respiratory flow for which the phase Φ is less than or equal to 0.5. A number of evenly spaced points (for example, sixty-five), representing points in time, are interpolated by an interpolator along the inspiratory flow-time curve for each breath. The curve described by the points is then scaled by a scaler to have unity length (duration/period) and unity area to remove the effects of changing respiratory rate and depth. The scaled breaths are then compared in a comparator with a pre-stored template representing a normal unobstructed breath. Breaths deviating by more than a specified threshold (typically 1 scaled unit) at any time during the inspiration from this template, such as those due to coughs, sighs, swallows and hiccups, as determined by a test element, are rejected. For non-rejected data, a moving average of the first such scaled point is calculated by central controller 4230 for the preceding several inspiratory events. This is repeated over the same inspiratory events for the second such point, and so on. Thus, for example, sixty five scaled data points are generated by central controller 4230, and represent a moving average of the preceding several inspiratory events, e.g., three events. The moving average of continuously updated values of the (e.g., sixty five) points are hereinafter called the “scaled flow”, designated as Qs(t). Alternatively, a single inspiratory event can be utilised rather than a moving average.

From the scaled flow, two shape factors relating to the determination of partial obstruction may be calculated.

Shape factor 1 is the ratio of the mean of the middle (e.g., thirty-two) scaled flow points to the mean overall (e.g., sixty-five) scaled flow points. Where this ratio is in excess of unity, the breath will be taken to be normal. Where the ratio is unity or less, the breath will be taken to be obstructed. A ratio of about 1.17 is taken as a threshold between partially obstructed and unobstructed breathing, and equates to a degree of obstruction that would permit maintenance of adequate oxygenation in a typical user.

Shape factor 2 is calculated as the RMS deviation from unit scaled flow, taken over the middle (e.g., thirty two) points. An RMS deviation of about 0.2 units is taken to be normal. An RMS deviation of zero is taken to be a totally flow-limited breath. The closer the RMS deviation to zero, the breath will be taken to be more flow limited.

Shape factors 1 and 2 may be used as alternatives, or in combination. In other forms of the present technology, the number of sampled points, breaths and middle points may differ from those described above. Furthermore, the threshold values can other than those described.

### 4.3.3.2.4 Determination of Apneas and Hypopneas

In one form of the present technology, a central controller 4230 executes one or more algorithms 4325 for the detection of apneas and/or hypopneas.

In one form, the one or more apnea/hypopnea detection algorithms 4325 receive as an input a respiratory flow rate Qr and provide as an output a flag that indicates that an apnea or a hypopnea has been detected.

In one form, an apnea will be said to have been detected when a function of respiratory flow rate Qr falls below a flow threshold for a predetermined period of time. The function may determine a peak flow, a relatively short-term mean flow, or a flow intermediate of relatively short-term mean and peak flow, for example an RMS flow. The flow threshold may be a relatively long-term measure of flow.

In one form, a hypopnea will be said to have been detected when a function of respiratory flow rate Qr falls below a second flow threshold for a predetermined period of time. The function may determine a peak flow, a relatively short-term mean flow, or a flow intermediate of relatively short-term mean and peak flow, for example an RMS flow. The second flow threshold may be a relatively long-term measure of flow. The second flow threshold is greater than the flow threshold used to detect apneas.

In one form, such respiratory events may be characterized as central or obstructive based at least in part on the aforementioned finger sensor PPG based type detection.

### 4.3.3.2.5 Detection of Snore

In one form of the present technology, a central controller 4230 executes one or more snore detection algorithms 4326 for the detection of snore.

In one form, the snore detection algorithm 4326 receives as an input a respiratory flow rate signal Qr and provides as an output a metric of the extent to which snoring is present.

The snore detection algorithm 4326 may comprise a step of determining the intensity of the flow rate signal in the range of 30-300 Hz. The snore detection algorithm 4326 may further comprises a step of filtering the respiratory flow rate signal Qr to reduce background noise, e.g., the sound of airflow in the system from the blower 4142.

### 4.3.3.2.6 Determination of Airway Patency

In one form of the present technology, a central controller 4230 executes one or more algorithms 4327 for the determination of airway patency.

In one form, airway patency algorithm 4327 receives as an input a respiratory flow rate signal Qr, and determines the power of the signal in the frequency range of about 0.75 Hz and about 3 Hz. The presence of a peak in this frequency range is taken to indicate an open airway. The absence of a peak is taken to be an indication of a closed airway.

In one form, the frequency range within which the peak is sought is the frequency of a small forced oscillation in the treatment pressure Pt. In one implementation, the forced oscillation is of frequency 2 Hz with amplitude about 1 cmH₂O.

In one form, airway patency algorithm 4327 receives as an input a respiratory flow rate signal Qr, and determines the presence or absence of a cardiogenic signal. The absence of a cardiogenic signal is taken to be an indication of a closed airway.

### 4.3.3.2.7 Determination of Therapy Parameters

In some forms of the present technology, the central controller 4230 executes one or more therapy parameter determination algorithms 4329 for the determination of one or more therapy parameters using the values returned by one or more of the other algorithms in the therapy engine module 4320.

In one form of the present technology, the therapy parameter is an instantaneous treatment pressure Pt. In one implementation of this form, the therapy parameter determination algorithm 4329 determines the treatment pressure Pt using the equation

$$Pt = A Π ( Φ ) + {P}_{0} \qquad ( 1 )$$

where:
A is an amplitude,
Φ is the current value of phase;
π(Φ) is the waveform template value (in the range 0 to 1) at the current value of phase, and
P₀ is a base pressure.

If the waveform determination algorithm 4322 provides the waveform template π(Φ) as a lookup table of values indexed by phase Φ, the therapy parameter determination algorithm 4329 applies equation (1) by locating the nearest lookup table entry to the current value Φ of phase returned by the phase determination algorithm 4321, or by interpolation between the two entries straddling the current value Φ of phase.

The values of the amplitude A and the base pressure P₀ may be set by the therapy parameter determination algorithm 4329 depending on the chosen pressure therapy mode in the manner described below.

### 4.3.3.3 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of gas whose mask pressure Pm at the patient interface 3000 is equal to the treatment pressure Pt.

### 4.3.3.4 Detection of Fault Conditions

In one form of the present technology, a processor executes one or more methods 4340 for the detection of fault conditions. The fault conditions detected by the one or more methods may include at least one of the following:
Power failure (no power, or insufficient power)
Transducer fault detection
Failure to detect the presence of a component
Operating parameters outside recommended ranges (e.g., pressure, flow, temperature, PaO₂)
Failure of a test alarm to generate a detectable alarm signal.

Upon detection of the fault condition, the corresponding algorithm signals the presence of the fault by one or more of the following:
Initiation of an audible, visual &/or kinetic (e.g., vibrating) alarm
Sending a message to an external device
Logging of the incident

### 4.4 Humidifier

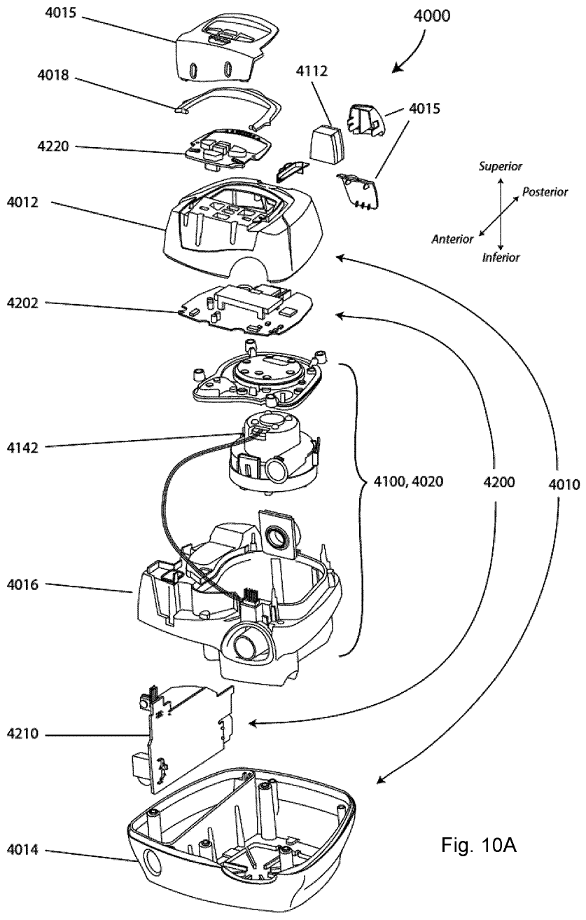

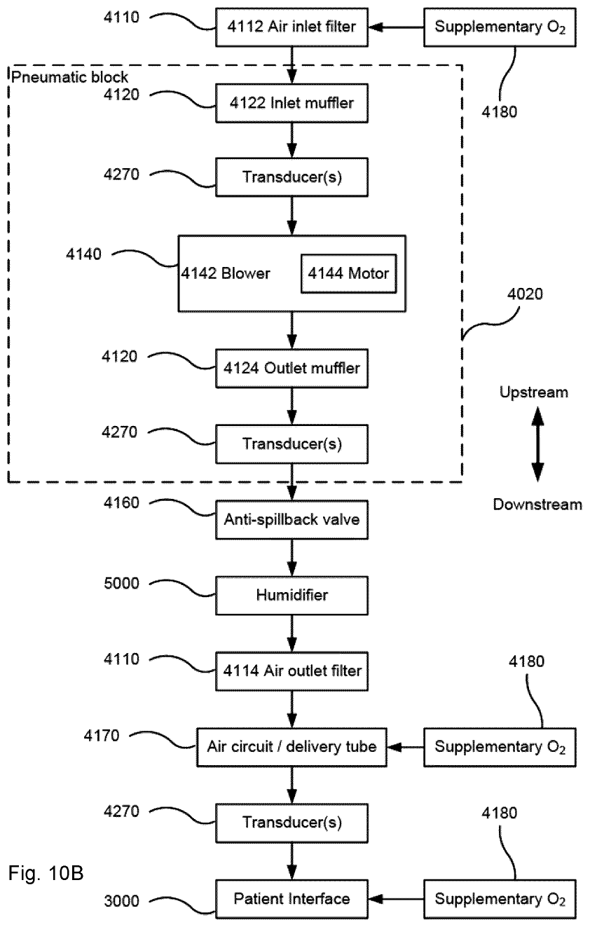

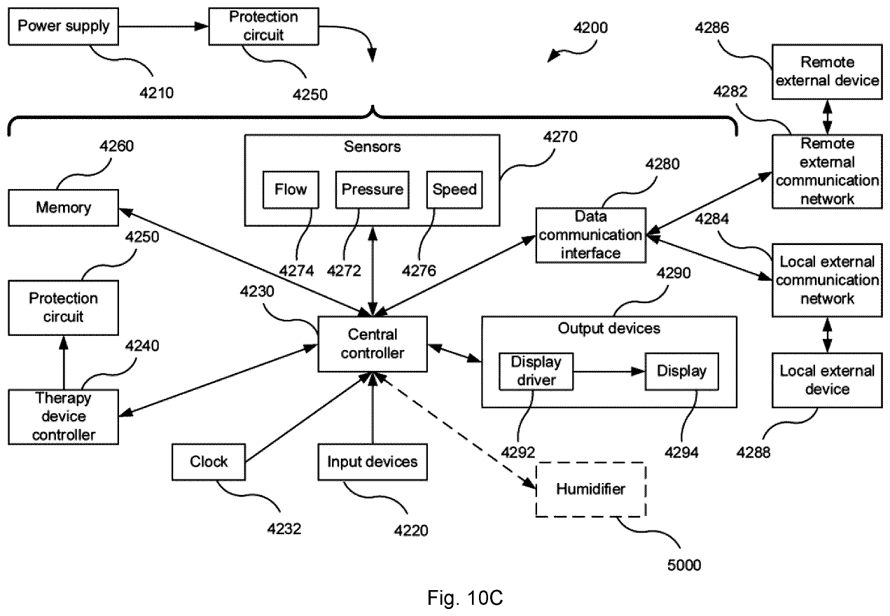

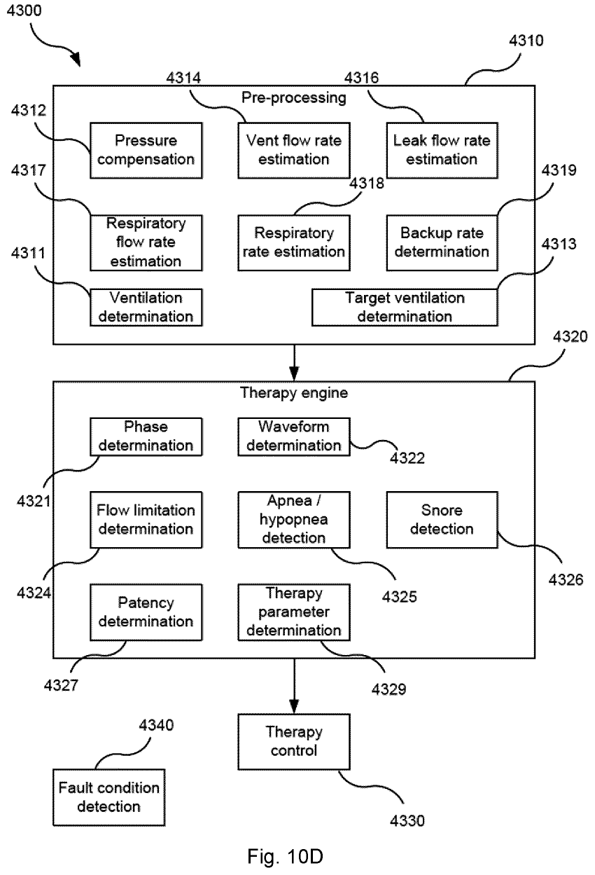

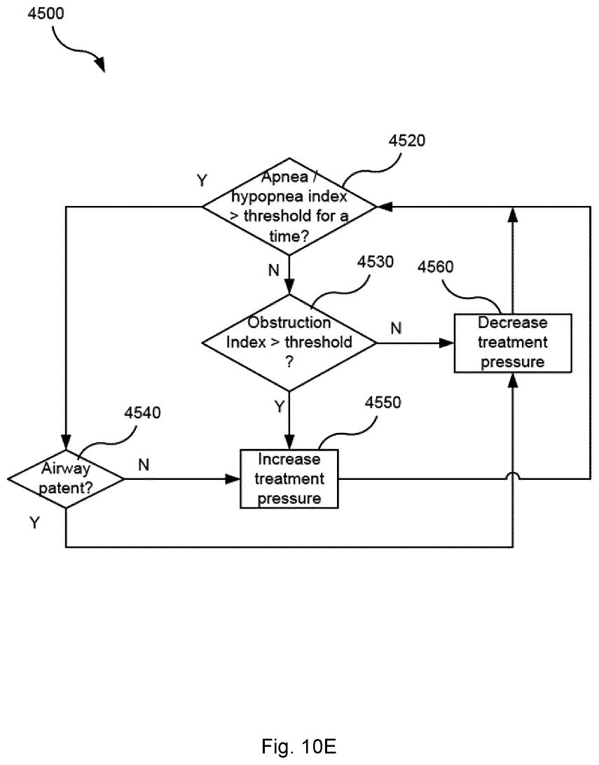

In one form of the present technology there is provided a humidifier 5000 (e.g., as shown in FIG. 10) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

### 4.5 Glossary

For the purposes of the present disclosure, in certain forms of the present technology, one or more of the following definitions may apply. In other forms of the present technology, alternative definitions may apply.

### 4.5.1 General

Air: In certain forms of the present technology, air may be taken to mean atmospheric air, and in other forms of the present technology air may be taken to mean some other combination of breathable gases, e.g., atmospheric air enriched with oxygen.

Respiratory Pressure Therapy (RPT): The delivery of a supply of air to the airways at a treatment pressure that is typically positive with respect to atmosphere.

Continuous Positive Airway Pressure (CPAP) therapy: Respiratory pressure therapy in which the treatment pressure is approximately constant through a breathing cycle of a patient. In some forms, the pressure at the entrance to the airways will be slightly higher during exhalation, and slightly lower during inhalation. In some forms, the pressure will vary between different breathing cycles of the patient, for example, being increased in response to detection of indications of partial upper airway obstruction, and decreased in the absence of indications of partial upper airway obstruction.

Patient: A person, whether or not they are suffering from a respiratory disease.

Automatic Positive Airway Pressure (APAP) therapy: CPAP therapy in which the treatment pressure is automatically adjustable, e.g., from breath to breath, between minimum and maximum limits, depending on the presence or absence of indications of SDB events.

### 4.5.2 Aspects of the Breathing Cycle

Apnea: According to some definitions, an apnea is said to have occurred when respiratory flow rate falls below a predetermined threshold for a duration, e.g., 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort.

Breathing rate, or respiratory rate (Rs): The rate of spontaneous respiration of a patient, usually measured in breaths per minute.

Duty cycle: The ratio of inhalation time, Ti to total breath duration, Ttot.

Effort (breathing): The work done by a spontaneously breathing person attempting to breathe.

Expiratory portion of a breathing cycle: The period from the start of expiratory flow to the start of inspiratory flow.

Flow limitation: The state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Hypopnea: A reduction in flow, but not a cessation of flow. In one form, a hypopnea may be said to have occurred when there is a reduction in flow below a threshold for a duration. In one form in adults, the following either of the following may be regarded as being hypopneas:
(i) a 30% reduction in patient breathing for at least 10 seconds plus an associated 4% desaturation; or
   (ii) a reduction in patient breathing (but less than 50%) for at least 10 seconds, with an associated desaturation of at least 3% or an arousal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed.

Positive End-Expiratory Pressure (PEEP): The pressure above atmosphere in the lungs that exists at the end of expiration.

Peak flow rate (Qpeak): The maximum value of flow during the inspiratory portion of the respiratory flow rate waveform.

Respiratory flow airflow rate, patient flow airflow rate (Qr): These synonymous terms may be understood to refer to the RPT device's estimate of respiratory airflow rate, as opposed to “true respiratory flow rate” or “true respiratory airflow rate”, which is the actual respiratory flow rate experienced by the patient, usually expressed in litres per minute.

Tidal volume (Vt): The volume of air inhaled or exhaled during normal breathing, when extra effort is not applied.

Inhalation Time (Ti): The duration of the inspiratory portion of the respiratory flow rate waveform.

Exhalation Time (Te): The duration of the expiratory portion of the respiratory flow rate waveform.

(total) Time, or breath duration (Ttot): The total duration between the start of the inspiratory portion of one respiratory flow rate waveform and the start of the inspiratory portion of the following respiratory flow rate waveform.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of the total amount of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 4.5.3 RPT Device Parameters

Flow rate: The instantaneous volume (or mass) of air delivered per unit time. While flow rate and ventilation have the same dimensions of volume or mass per unit time, flow rate is measured over a much shorter period of time. Flow may be nominally positive for the inspiratory portion of a breathing cycle of a patient, and hence negative for the expiratory portion of the breathing cycle of a patient. In some cases, a reference to flow rate will be a reference to a scalar quantity, namely a quantity having magnitude only. In other cases, a reference to flow rate will be a reference to a vector quantity, namely a quantity having both magnitude and direction. Flow rate will be given the symbol Q. ‘Flow rate’ is sometimes shortened to simply ‘flow’. Total flow rate, Qt, is the flow of air leaving the RPT device. Vent flow rate, Qv, is the flow of air leaving a vent to allow washout of exhaled gases. Leak flow rate, Ql, is the flow rate of unintentional leak from a patient interface system. Respiratory flow rate, Qr, is the flow of air that is received into the patient's respiratory system.

Leak: The word leak will be taken to be an unintended flow of air. In one example, leak may occur as the result of an incomplete seal between a mask and a patient's face. In another example leak may occur in a swivel elbow to the ambient.

Pressure: Force per unit area. Pressure may be measured in a range of units, including cmH₂O, g-f/cm², hectopascal. 1 cmH₂O is equal to 1 g-f/cm² and is approximately 0.98 hectopascal. In this specification, unless otherwise stated, pressure is given in units of cmH₂O. The pressure in the patient interface (mask pressure) is given the symbol Pm, while the treatment pressure, which represents a target value to be achieved by the mask pressure Pm at the current instant of time, is given the symbol Pt.

### 4.5.4 Terms for Ventilators

Adaptive Servo-Ventilator (ASV): A servo-ventilator that has a changeable rather than a fixed target ventilation. The changeable target ventilation may be learned from some characteristic of the patient, for example, a respiratory characteristic of the patient.

Backup rate: A parameter of a ventilator that establishes the respiratory rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired mask pressure which the ventilator will attempt to achieve at a given time.

End expiratory pressure (EEP): Desired mask pressure which the ventilator will attempt to achieve at the end of the expiratory portion of the breath. If the pressure waveform template π(Φ) is zero-valued at the end of expiration, i.e., π(Φ)=0 when Φ=1, the EEP is equal to the EPAP.

IPAP: desired mask pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP-EPAP). In some contexts pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Servo-assistance: Pressure support minus minimum pressure support.

Spontaneous Timed (S T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the inspiratory portion of the breathing cycle by the patient's efforts.

Typical recent ventilation: The typical recent ventilation Vtyp is the value around which recent measures of ventilation over some predetermined timescale tend to cluster, that is, a measure of the central tendency of the measures of ventilation over recent history.

Ventilator: A mechanical device that provides pressure support to a patient to perform some or all of the work of breathing.

### 4.5.5 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 4.6 Other Remarks

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being preferably used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated by reference to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

Moreover, in interpreting the disclosure, all terms should be interpreted in the broadest reasonable manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular embodiments, it is to be understood that these embodiments are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative embodiments and that other arrangements may be devised without departing from the spirit and scope of the technology.

Although the present invention has been illustrated by reference to specific embodiments, it will be apparent to those skilled in the art that the invention is not limited to the details of the foregoing illustrative embodiments, and that the present technology may be embodied with various changes and modifications without departing from the scope thereof. The present examples are therefore to be considered in all respects as illustrative and not restrictive, the scope of the technology being indicated by the appended claims rather than by the foregoing description, and all changes which come within the meaning and range of equivalency of the claims are therefore intended to be embraced therein. In other words, it is contemplated to cover any and all modifications, variations or equivalents that fall within the scope of the basic underlying principles and whose essential attributes are claimed in this patent application. It will furthermore be understood by the reader of this patent application that the words “comprising” or “comprise” do not exclude other elements or steps, that the words “a” or “an” do not exclude a plurality, and that a single element, such as a computer system, a processor, or another integrated unit may fulfil the functions of several means recited in the claims. Any reference signs in the claims shall not be construed as limiting the respective claims concerned. The terms “first”, “second”, third”, “a”, “b”, “c”, and the like, when used in the description or in the claims are introduced to distinguish between similar elements or steps and are not necessarily describing a sequential or chronological order. Similarly, the terms “top”, “bottom”, “over”, “under”, and the like are introduced for descriptive purposes and not necessarily to denote relative positions. It is to be understood that the terms so used are interchangeable under appropriate circumstances and embodiments of the technology are capable of operating according to the present technology in other sequences, or in orientations different from the one(s) described or illustrated above.

### FURTHER EXAMPLES OF THE TECHNOLOGY

The following paragraphs further illustrate examples of the present technology described herein.

Example 1. A computer-implemented method for detecting sleep disordering events, the method comprising the steps of:
obtaining at least one physiological signal;
identifying from the at least one physiological signal seed events indicatory of sleep disordering events;
determining substantially regular patterns within at least part of the seed events;
determining degrees of fit of the seed events to the substantially regular patterns;
detecting the sleep disordering events by selecting seed events based on the determined degrees of fit.

Example 2. The method of Example 1, wherein the at least one physiological signal is at least one of a PAT signal, an oxygen saturation signal, a pulse rate signal, a respiratory effort signal, and an airflow signal.

Example 3. The method of any of the preceding Examples, wherein the seed events are at least one of a PAT signal amplitude drop from a baseline, a desaturation of oxygen, a pulse rate signal amplitude increase from a baseline, a respiratory effort signal amplitude decrease from a baseline, and an airflow signal amplitude decrease from a baseline.

Example 4. The method of any of the preceding Examples, wherein the substantially regular patterns includes a substantially regular morphological pattern.

Example 5. The method of any of the preceding claims, wherein the substantially regular patterns includes a substantially regular temporal pattern.

Example 6. The method of any of the preceding Examples, wherein the step of determining the substantially regular patterns and the step of determining degrees of fit includes at least one of determining a duration of a seed event, determining an intensity of a seed event, deriving a morphological asymmetry and/or characterizing a morphological shape of a seed event.

Example 7. The method of any of the preceding Examples, wherein the step of determining the substantially regular patterns and the step of determining degrees of fit includes at least one of determining a starting point of the seed event, determining an end point of the seed event, determining a point of highest or lowest intensity and/or determining any other characteristic point of a seed event.

Example 8. The method according to any of the preceding Examples, wherein the step of detecting sleep disordering events includes using a classifier trained or developed for said detecting.

Example 9. The method according to any of the preceding Examples, wherein a plurality of physiological signals spanning a common time span which includes seed events is obtained.

Example 10. The method according to claim 9, further comprising the step of grouping seed events from the plurality of physiological signals into sets, wherein the seed events grouped into the set are indicatory of the same sleep disordering event, wherein the grouping of seed events into the set includes determining a substantially regular pattern in a co-occurrence of the seed events of the set.

Example 11. The method according to Example 8 and Example 10, wherein the classifier is configured to detect within the sets of seed events the respiratory related sleep disordering events, or the non-respiratory related sleep disordering events.

Example 12. The method according to any of the preceding Examples, further comprising a step of feedback on a detection of a sleep disordering event or not by a selection of at least one seed event, wherein the feedback is preferably rendered in natural language.

Example 13. A controller comprising at least one processor and at least one memory including computer program code, the at least one memory and computer program code configured to, with the at least one processor, cause the controller to perform a method according to any of the preceding Examples 1-12.

Example 14. A computer program product comprising computer-executable instructions for performing the method according to any of the preceding Examples 1-12 when the program is run on a computer.

Example 15. A computer readable storage medium comprising computer-executable instructions for performing the method according to any of the preceding Examples 1-12 when the program is run on a computer.

1. A processor-implemented method for detecting sleep disordering events, the method comprising:
accessing a plurality of physiological signals generated by one or more sensors;
detecting, from the plurality of physiological signals, seed events suggestive of sleep disordering events;
computing features indicative of patterns within portions of the plurality of physiological signals that are associated with the detected seed events;
applying, to a classifier, the computed features indicative of patterns of the seed events, wherein the classifier is trained to compute a degree of fit of the computed features to learned patterns of sleep disordering events; and
outputting an identification of one or more sleep disordering events corresponding with the seed events based on the computed degree of fit determined by the classifier.

2. The method of claim 1 wherein the classifier comprises one or more of a machine learning classifier, a decision tree model, a machine learning classifier model, a logistic regression classifier model, a neural network, Naive Bayes classifier model, and a support vector machine.

3. The method of any one of claims 1 to 2, wherein the plurality of physiological signals comprise a peripheral arterial tone (PAT) signal, and one or more of: an oxygen saturation signal, a pulse rate signal, a respiratory effort signal, a movement signal, and an air flow signal.

4. The method of any one of claims 1 to 3, wherein each seed event of the seed events comprises one or more of: an amplitude drop from a baseline within a peripheral arterial tone (PAT) signal, a desaturation of oxygen in an oxygen saturation (SpO2) signal, an amplitude increase from a baseline in a pulse rate (PR) signal, an amplitude change from a baseline in a respiratory effort signal, and an amplitude change from a baseline in air flow rate signal.

5. The method of any one of claims 1 to 4, wherein the patterns include a morphological pattern.

6. The method of any one of claims 1 to 5, wherein the patterns include a temporal pattern.

7. The method of any one of claims 1 to 6 wherein a feature of the computed features comprises one or more of: a duration of a seed event; an intensity of a seed event; a derived inclination or slope of a seed event; a derived morphological asymmetry of changing slope of a vicinity of a seed event; a depth of a seed event; a variance of the signal amplitudes of the seed event; an average of the signal amplitudes of the seed event; a skewness of the seed event; and a characterization of a morphological shape of a seed event.

8. The method of any one of claims 1 to 7, wherein one or more features of the computed features comprises one or more of a determined starting point of the seed event, a determined end point of the seed event, a determined point of highest or lowest intensity, and a determined characteristic point of a seed event.

9. The method according to any one of claims 1 to 8 wherein the detected seed events comprise a first seed event of a first signal of the plurality of physiological signals and a second seed event of a second signal of the plurality of physiological signals, wherein the second signal is a different physiological signal from the first signal, and wherein one or more features of the computed features characterize the first seed event and the second seed event.

10. The method according to claim 9 wherein the one or more features of the computed features that characterize the first seed event and the second seed event comprises: (a) a time amount that a desaturation nadir trails or precedes a peak pulse rate increase and/or a PAT signal amplitude decrease; and/or (b) a timing difference between a detected pulse rate (PR) surge peak and a decrease valley of a PAT signal.

11. The method according to claim 9, wherein the detected seed events comprise a third seed event of the first signal, and wherein one or more features of the computed features characterizes the first seed event and the third seed event.

12. The method of claim 11 wherein the first seed event and the third seed event comprise a neighboring pair of seed events.

13. The method of any one of claims 11 to 12 wherein the one or more features of the computed features that characterizes the first seed event and the third seed event comprises any one or more of: (a) a duration between the first seed event and the third seed event; (b) a computed stability of a period between the first seed event and the third seed event; and (c) a computed stability of the first seed event and the third seed event.

14. The method of claim 13 wherein computing the duration comprises detection of a characteristic point in each of the first seed event and the third seed event and determining the duration based on an interval associated with the detected characteristic points.

15. The method of claim 14 wherein the detected characteristic points comprise one or more of a local amplitude minimum and local amplitude maximum.

16. The method of any one of claims 13 to 15 wherein the computed stability is derived from a plurality of seed events and comprises one or more of a depth, average, and a variance.

17. The method according to claim 11 wherein the detected seed events comprise a fourth seed event of the second signal, wherein one or more features of the computed features characterizes (a) the first seed event and the third seed event of the first signal and (b) the second seed event and the fourth seed event of the second signal.

18. The method of claim 17 wherein the one or more features of the computed features that characterizes (a) the first seed event and the third seed event of the first signal and (b) the second seed event and the fourth seed event of the second signal comprises: a temporal correspondence of (a) detected pulse rate (PR) peaks of seed events of a PR signal, and (b) reduction in peripheral arterial tone (PAT) to a minimum point in valleys of seed events of a PAT signal.

19. The method of any one of claims 1 to 18 further comprising generating the outputting of the identification as feedback in response to a user input of a selection, on user interface, of at least one seed event detected by the detecting implemented by one or more processors.

20. The method of any one of claims 1 to 19 further comprising generating a signal for controlling operation of a respiratory therapy apparatus based on the outputting or the applying.

21. The method of claim 20 wherein the generating comprises transmitting the identification of the one or more sleep disordering events to a remote computing system or server.

22. The method of any one of claims 20 to 21 wherein the generating comprises transmitting the signal to the respiratory therapy apparatus via a network communications link.

23. A controller comprising at least one processor and at least one memory including processor control instructions, the at least one memory and processor control instructions configured to, with the at least one processor, cause the controller to perform a method according to any one of claims 1 to 22.

24. Apparatus for detecting sleep disordering events, the apparatus comprising:
one or more sensors;
a controller comprising one or more processors and at least one memory including processor control instructions;
wherein the controller is configured to:
access a plurality of physiological signals generated by one or more sensors;
detect, from the plurality of physiological signals, seed events suggestive of sleep disordering events;
compute features indicative of patterns within portions of the plurality of physiological signals that are associated with the detected seed events;
apply, to a classifier, the computed features indicative of patterns of the seed events, wherein the classifier is trained to compute a degree of fit of the computed features to learned patterns of sleep disordering events; and
output an identification of one or more sleep disordering events corresponding with the seed events based on the computed degree of fit determined by the classifier.

25. A processor-readable storage medium comprising processor-executable instructions for performing the method according to any of claims 1-22 when executed by one or more processors.

26. A processor-readable medium, having stored thereon processor-executable instructions which, when executed by one or more processors, cause the one or more processors to detect sleep disordering events, the processor-executable instructions comprising:
instructions to access a plurality of physiological signals generated by one or more sensors;
instructions to detect, from the plurality of physiological signals, seed events suggestive of sleep disordering events;
instructions to compute features indicative of patterns within portions of the plurality of physiological signals that are associated with the detected seed events;
instructions to apply, to a classifier, the computed features indicative of patterns of the seed events, wherein the classifier is trained to compute a degree of fit of the computed features to learned patterns of sleep disordering events; and
instructions to output an identification of one or more sleep disordering events corresponding with the seed events based on the computed degree of fit determined by the classifier.

27. The processor-readable medium of claim 26, wherein the processor-executable instructions further comprise instructions to generate a signal for controlling operation of a respiratory therapy apparatus based on the outputting or applying.

28. The processor-readable medium of claim 27, wherein the controlling operation comprises controlling a pressure or flow therapy of a blower of the respiratory therapy apparatus.

29. A server with access to the processor-readable medium of any one of claims 25 to 28, wherein the server is configured to receive requests for downloading the processor-executable instructions of the processor-readable medium to a processing device over a network.

30. A processing device comprising: one or more processors; and (a) a processor-readable medium of any one of claims 25 to 28, or (b) wherein the processing device is configured to access the processor-executable instructions with the server of claim 29.

31. The processing device of claim 30, wherein the processing device is a respiratory therapy apparatus.

32. The processing device of claim 31, wherein the processing device is configured to generate a pressure therapy or a flow therapy.

33. A method of a server having access to the processor-readable medium of any one of claims 25 to 28, the method comprising receiving, at the server, a request for downloading the processor-executable instructions of the processor-readable medium to an electronic processing device over a network; and transmitting the processor-executable instructions to the electronic processing device in response to the request.

34. A method of one or more processors for detecting sleep disordering breathing events, comprising:
accessing, with the one or more processors, the processor-readable medium of any one of claims 25 to 28, and
executing, in the one or more processors, the processor-executable instructions of the processor-readable medium.
